(19) United States
(12) Patent Application Publication
(10) Pub. No.: US 2022/0331592 A1
(43) Pub. Date: Oct. 20, 2022

(54) Neurostimulation Device for Treating Pelvic Pain and External Genital Pain in Female Patients

(71) Applicant: IVES, LLC, Annapolis, MD (US)

(72) Inventor: Erik B. YOUNG, Annapolis, MD (US)

(73) Assignee: IVES, LLC, Annapolis, MD (US)

(21) Appl. No.: 17/504,724

(22) Filed: Oct. 19, 2021

Related U.S. Application Data
(60) Provisional application No. 63/177,222, filed on Apr. 20, 2021.

(51) Int. Cl. A61N 1/36; A61N 1/04; A61N 1/05

(52) U.S. Cl. CPC A61N 1/36071; A61N 1/0456; A61N 1/0524

(57) ABSTRACT

Embodiments of the present invention provide an intravaginal electrical neurostimulation device for treating pelvic pain and external genital pain in female patients capable of delivering targeted electrical stimulation to the pelvic nerves, the paracervical nerves and the sacral nerves using an electrode that is in direct contact with the paracervical vaginal epithelium in the posterior and lateral vaginal fornices. Beneficially, devices constructed and used in accordance with embodiments of the present invention can be used without the aid of a medical practitioner and can be dynamically controlled and adjusted by the patient to provide personalized electrical stimulation patterns when the patient is experiencing pain or when she anticipates the onset of pelvic pain due to, for example, menstruation or sexual intercourse. Embodiments of the present invention typically comprise a set of intravaginal components and a set of extravaginal components. The set of intravaginal components include a frame, a paracervical electrode (or electrodes), and a paracervical electrode connecting wire (or wires) that exit the vagina through the vaginal orifice. The set of extravaginal components comprises the IVENS Stimulator (IS), an electrical stimulation generator, an optional external controller, a cutaneous electrode (or electrodes), a cutaneous electrode connecting wire (or wires), and the portion of the paracervical electrode connecting wire or wires that are outside of the vagina.

### FIELD OF THE INVENTION

Embodiments of the present invention relate generally to methods and devices for treating pelvic pain and external genital pain in women, and more particularly to methods and devices that use electrical stimulation to cause neuromodulation of the nerves that transmit the sensation of pain from anatomical structures in the pelvis and the external genitalia to the central nervous system in women.

### BACKGROUND OF THE INVENTION

Many women suffer from pelvic pain. Types of pelvic pain include, without limitation, dysmenorrhea, dyspareunia, chronic pelvic pain, pain originating in the urinary tract, myofascial pain originating in the muscles and supporting tissues of the pelvis, pain related to childbirth, post-surgical pain and idiopathic pain. In addition, some women experience pain in the area of their external genitalia.

Pelvic pain in women typically originates in their reproductive organs, bladder, pelvic floor or the peritoneal lining of the pelvis. The sensation of pelvic pain is transmitted to the central nervous system through sympathetic, parasympathetic, and afferent sensory nerve fibers that traverse nerves and nerve plexuses in the abdomen and the pelvis, and the thoracic, lumbar, and sacral nerves. The sensation of pain originating in the external genitalia is transmitted to the central nervous system via afferent sensory fibers that traverse the pudendal nerves and the S2, S3 and S4 sacral nerve roots.

The sensation of pelvic pain from the intraperitoneal reproductive organs (e.g., uterus, ovaries and fallopian tubes) reaches the central nervous system by way of the hypogastric nerves, superior hypogastric plexus, lower aortic plexus, ovarian plexus, lumbar splanchnic nerves, lower thoracic and lumbar nerves, uterovaginal plexuses and the inferior hypogastric plexuses. The sensation of pelvic pain from the pelvic floor and the subperitoneal reproductive organs (e.g., the cervix and upper vagina) reaches the central nervous system by way of the uterovaginal plexuses, inferior hypogastric plexuses, pelvic splanchnic nerves and the S2, S3 and S4 nerves. The sensation of pelvic pain from the lower vagina and the external genitalia reaches the central nervous system by way of the pudendal nerves and the S2, S3 and S4 nerves.

For the purposes of this disclosure, the nerves that innervate the intraperitoneal reproductive organs and traverse the hypogastric nerves, superior hypogastric plexus, lower aortic plexus, ovarian plexus, lumbar splanchnic nerves, lower thoracic and lumbar nerves shall hereinafter be referred to collectively as the “pelvic nerves.” The nerves that innervate the intraperitoneal reproductive organs and the subperitoneal reproductive organs and traverse the uterovaginal plexuses, inferior hypogastric plexuses, pelvic splanchnic nerves and the S2, S3 and S4 nerves shall hereinafter be referred to collectively as the “paracervical nerves.” The nerves that innervate the lower vagina and the external genitalia and traverse the pudendal nerves and the S2, S3 and S4 nerves shall hereinafter be referred to collectively as the “sacral nerves.”

It should be noted that uterosacral plexuses and the inferior hypogastric plexuses are located in the areas where the uterosacral ligaments and the cardinal ligaments attach to each side of the cervix. These areas are located just above (superior to and internal to) the areas where the vaginal epithelium joins the cervix in the posterior and lateral fornices on each side of the midline. A significant concentration of sympathetic, parasympathetic and afferent sensory nerves and nerve fibers related to the “paracervical nerves” (as defined above) are present in these areas.

Electrical neuromodulation devices (sometimes referred to as electrical stimulation devices) have been developed to deliver electrical stimulation to specific collections of neurons in the brain, the spine, and a sacral nerve root. These devices usually use an electrical pulse generator to deliver pulsed electrical current to electrodes that are placed in close proximity to these specific collections of neurons in the brain, spinal cord or sacral nerve root to neuromodulate the nearby neurons or nerves.

To date, however, no electrical neuromodulating device has been developed that is capable of delivering targeted electrical stimulation to the pelvic nerves, the paracervical nerves and the sacral nerves. Moreover, prior to the present invention, no electrical neuromodulation device has been developed that delivers electrical current to an electrode that is placed inside a woman's upper vagina. Furthermore, prior to the present invention, no electrical neuromodulating device provides an electrode that is in contact with the paracervical vaginal epithelium in the posterior and lateral vaginal fornices to deliver targeted electrical stimulation to cause neuromodulation of the pelvic nerves, the paracervical nerves and the sacral nerves.

The ability of existing electrical stimulation devices to reduce or relieve pelvic pain and external genital pain is extremely limited because the pelvic nerves, paracervical nerves and sacral nerves all reside deep within the pelvis. Consequently, there is considerable need for an intravaginal device that places a paracervical electrode or electrodes in a woman's vagina without surgery, and which can deliver targeted electrical stimulation to the pelvic nerves, the paracervical nerves and the sacral nerves.

### SUMMARY OF THE INVENTION

Embodiments of the present invention address the aforementioned need by providing an intravaginal electrical neurostimulation device for treating pelvic pain and external genital pain in female patients (the “IVENS device”) capable of delivering targeted electrical stimulation to the pelvic nerves, the paracervical nerves and the sacral nerves. More specifically, IVENS devices constructed according to embodiments of the present invention deliver targeted electrical stimulation to neuromodulate the pelvic nerves, the paracervical nerves and the sacral nerves using an electrode that is in direct contact with the paracervical vaginal epithelium in the posterior and lateral vaginal fornices. Beneficially, IVENS devices constructed and used in accordance with embodiments of the present invention can be used without the aid of a medical practitioner and can be dynamically controlled and adjusted by the patient to provide personalized electrical stimulation patterns when the patient is experiencing pain or when she anticipates the onset of pelvic pain due to, for example, menstruation or sexual intercourse.

In general, embodiments of the present invention typically comprise a set of intravaginal components and a set of extravaginal components. The intravaginal components include a frame, a paracervical electrode (or electrodes), and a paracervical electrode connecting wire (or wires) that exit the vagina through the vaginal orifice. The paracervical electrode comprises one or more wires embedded in the covering of the proximal portion of the frame that are capable of carrying or conducting electrical current. A “full-length” paracervical electrode is an electrode that extends entirely across the proximal portion of the frame. A “partial-length” paracervical electrode is a shorter electrode, embedded in the covering of the lateral and distal portions of the proximal portion of the frame. Paracervical electrode connecting wires are insulated wires that electrically connect a paracervical electrode to the IVENS Stimulator (described below). If the device is configured to use multiple paracervical electrodes, then that device will typically require multiple paracervical electrode connecting wires.

When the frame is properly positioned in the vagina, a full-length paracervical electrode will be in contact with the vaginal epithelium in the posterior and lateral vaginal fornices. If the frame is embedded with two partial-length paracervical electrodes with an electrode on each side of the frame, each of the partial-length paracervical electrodes will be in contact with the vaginal epithelium only in the lateral vaginal fornices on each side of the cervix.

The extravaginal components of the present invention include an IVENS Stimulator, and, in some embodiments, a cutaneous electrode (or electrodes), a cutaneous electrode connecting wire (or wires) and the portion of the paracervical electrode connecting wire or wires that extend through the vaginal orifice to remain on the outside of the vagina. The cutaneous electrode is an electrode that can conduct an electrical current that is attached to the patient's skin. The cutaneous electrode connecting wire is an insulated wire that electrically connect a cutaneous electrode to the IVENS Stimulator. If the device is configured to use multiple cutaneous electrodes, then that device will suitably require multiple cutaneous electrode connecting wires.

For the sake of brevity and ease of comprehension, full length paracervical electrodes, partial-length paracervical electrodes and cutaneous electrodes may hereinafter be referred to collectively as “electrodes,” unless the context dictates otherwise. Also, for the sake of brevity and ease of comprehension, the paracervical electrode connecting wires and cutaneous electrode connecting wires may be referred to collectively as “connecting wires,” unless the context dictates otherwise.

The IVENS Stimulator (IS) comprises an electrical stimulation generator (“ESG”) and at least one IVENS Stimulator Channel (“ISC”). Each ISC is configured with a pair of electrical connections to receive and electrically connect to a pair of connecting wires which are each connected to an electrode.

The ESG is configured to periodically generate and output electrical stimulation pulses to an ISC, which transmits the pulses to the pair of connecting wires attached to the ISC, and the electrodes attached to each connecting wire. The ESG, the electrical contacts in an ISC, the connecting wires attached to the electrical contacts in the ISC, and the electrodes attached to the connecting wires are the components of an Electrical Stimulation Circuit (“ESC”). When the IS is turned on and energized, electrical stimulation pulses from the ESG, create an electric potential between the electrodes in the ESC to create an electrical field between and around the electrodes in the ESC. The electrical field passes through and neuromodulates the pelvic nerves, paracervical nerves, and sacral nerves present within the electrical field to eliminate or reduce pelvic pain and external genital pain.

In some embodiments, the IS may be equipped with multiple ISCs. With multiple ISCs, embodiments of the present invention may be configured to produce multiple combinations of overlapping electrical fields produced by multiple electrical stimulation circuits that are designed to neuromodulate the pelvic nerves, the paracervical nerves, and the sacral nerves simultaneously, consecutively or in an alternating pattern of electrical fields. The overlapping electrical fields may (or may not) be tuned to have different characteristics, depending on the desired overall effect of applying the overlapping electrical fields to the pelvic nerves, the paracervical nerves and the sacral nerves.

The IS typically comprises a case with a digital display screen. The case, which is preferably constructed from a rigid or hard plastic material, has a hollow that defines an internal cavity for housing most of the electronic components of the IS. The case is preferably designed to be carried in one of the patient's pockets, or clipped or fastened to the woman's clothing in the area of her lower anterior abdominal wall (e.g., around her waist, on her hips, or in any of the areas where her pockets, if any, would normally be located). This positioning will allow the paracervical electrode connecting wire (or wires) and the cutaneous electrode connecting wire (or wires), if any, to be easily connected to the IS while the IS is carried in a pocket or attached to a convenient location on the woman's clothing. In other embodiments, the IS may come with, and/or be attached to a waist or shoulder strap.

Suitably, in addition to the electronic stimulation generator (ESG), the electronic components housed inside the case of the IS also include a printed circuit board, which is affixed to an interior wall of the case. Attached to the printed circuit board is an electronic circuit comprising a battery, a microprocessor, a memory, a local control program in the memory, a radio frequency transceiver and the aforementioned ESG. The local control program stored in the memory comprises one or more programming modules having programming instructions that, when executed by the microprocessor, will cause the microprocessor to perform certain functions herein described, including sending electronic signals to the ESG, and thereby controlling the output of the ESG. The ESG then converts the DC current from the battery into the appropriate electrical stimulation patterns (“ESP's”) to be delivered to one or more ISCs to create one or more electrical stimulation circuits to neuromodulate the pelvic nerves, the paracervical nerves and the sacral nerves.

The characteristics of the electrical stimulation produced by the ESG may be varied by, for example, varying the flow of current, varying the voltage, varying the frequency of the pulses, by using tonic stimulation or burst stimulation, or by altering the pulse width, frequency and amplitude of the electrical stimulation being produced. The operations of the microprocessor within the device may be controlled or adjusted by pressing buttons, or by flipping or sliding switches, on the external surface of the case of the IS. These physical (or iconic) buttons and/or switches, may be configured, for example, to permit the user to activate, deactivate or tune the ESG inside the IS.

In some embodiments of the present invention, the ESG inside the IS may be more dynamically (or more precisely) controlled by instructions and commands generated on an IVENS external controller (the “IEC”). The IEC may comprise, for example, a smart phone, tablet, tablet computer, a personal wearable device (such as a smart watch) or personal computer. In this embodiment, a control application executing on the IEC communicates with an IVENS device control application (the “IDCA”) executing in the primary memory of the IS. The communication is typically implemented between a first radio frequency transceiver located in the IEC communicating wirelessly with a second radiofrequency transceiver located in the IS using a standard wireless communication protocol, such as Bluetooth. More specifically, the first radio frequency transceiver inside the IEC may be configured to establish a wireless communication channel with the second radio frequency transceiver on the printed circuit board inside the IS.

The IEC also comprises a microprocessor, a memory, and another control application (referred to hereinafter as the “IVENS app”) that is stored in the memory of the IEC. The IVENS App comprises program instructions that, when executed by the microprocessor on the IEC, will cause the microprocessor on the IEC to communicate with the IDCA of the IS via the wireless data communications channel established between the radio frequency transceiver on the IS and the radio frequency transceiver on the IEC.

A user interface module in the IVENS app running on the IEC is configured to receive operating instructions from the user, and thereby permits a user to activate, adjust and tune the electrical stimulation being delivered by electrical stimulation generator in the IS to the electrodes in an electrical stimulation circuit. Thus, the user can manipulate controls on a display screen of the IEC (such as digital representations of buttons, icons and sliders) in the user interface on the IEC to select, personalize, optimize, adjust, activate and/or deactivate the electrical stimulation provided to the pelvic nerves, the paracervical nerves and the sacral nerves by the IVENS device. In addition, the radio frequency transceiver and the microprocessor inside the IEC can receive over the wireless communication channel IVENS device status information and other data generated by the microprocessor connected to the circuit board inside the IS case, and/or data stored in the memory on the circuit board inside the IS case. The status information and other data may be displayed on the display screen associated with the IEC via the user interface. Preferably, the user interface module in the IVENS app running on the IEC also includes program instructions configured to permit the IEC or the user to use email, text messaging or another data or information transmitting process to send the status information and other data retrieved from the memory of the IS to other devices, organizations or people, such as, for example, the user's personal physician or other health care provider.

Preferably, the IVENS app stored in the memory of the IEC also includes program instructions that permit the IEC to periodically query a remote computer system or server for (1) the availability of program updates associated with the control application running on the IEC, and/or (2) the availability of operating system updates, local program updates or firmware updates associated with the local control program stored in the memory of the IS. If such an update is available, the IVENS app may be configured to automatically download and install the update on the IEC, on the IS, and/or both. By downloading such updates as they become available, the IVENS app running on the IEC, as well as the operating system, application program and firmware running on the IS, will automatically remain substantially up-to-date with the latest bug fixes and/or improvements. In some embodiments, the user interface module of the IVENS app may be configured to prompt the user for permission or confirmation before downloading and/or installing new, updated or upgraded programs, operating systems or firmware.

### BRIEF DESCRIPTION OF DRAWINGS

FIG. 1 shows a diagram illustrating the conventional terms used to identify and describe the parts and planes of the human body, and the relationships therebetween.

FIG. 2 shows an illustration of a midline sectional view a human female pelvis.

FIG. 3 shows an illustration of a superior view of the pelvic viscera.

FIG. 4 shows an illustration of a superior view of the female pelvic floor with the peritoneum and uterus removed.

FIG. 5 shows a schematic representation of the nervous innervation of the female reproductive organs.

FIG. 6A is an enlarged view of thy: nervous intervention of the female reproductive organs.

FIG. 6B shows the location of the paracervical nerves on each side of the cervix within the oval surrounding the cervix.

FIG. 7 is a midline sagittal view of a female pelvis that shows the approximate location of the superior hypogastric plexus, which lies over the bifurcation of the aorta in the presacral space anterior to the sacral promontory, and the level on the uterine cervix where the paracervical nerves are concentrated on each side of the cervix, lateral to the midline.

FIG. 8 is a superior view of the pelvis viscera showing the areas where the of paracervical nerves are concentrated in the areas where the uterosacral and cardinal ligaments attach to each side of the cervix and the location of the superior hypogastric ganglion.

FIG. 9 is a superior view of the pelvic floor with the uterus and perineum removed showing the areas where the of paracervical nerves are concentrated where the uterosacral and cardinal ligaments attach to each side of the cervix and the location of the superior hypogastric ganglion.

FIG. 10 is a schematic diagram illustrating in more detail the primary components of one embodiment of the IVENS device.

FIG. 11 shows a detailed top view of the primary intravaginal components of one embodiment of the IVENS device.

FIG. 12 shows a perspective view of the intravaginal components of the IVENS device as seen from a left, distal and anterior-lateral point of view.

FIG. 13 shows a perspective view of the intravaginal components of the IVENS device as seen from a left, proximal and anterior-lateral point of view.

FIGS. 14, 15, 16 and 17 show schematic diagrams illustrating the typical placement and orientation of the frame within the vagina in accordance with some embodiments of the present invention.

FIGS. 18A and 18B show a top view and a side view illustration of a full length paracervical electrode embedded in the covering of the proximal portion of the frame respectively.

FIG. 19 shows a representation of a female patient wearing an IVENS device showing the approximate location of the intravaginal and extravaginal components of the IVENS device required by the different embodiments of the device

FIG. 20 shows an intrapelvic view of the electrical field created by the first mode of operation for the first embodiment of the present invention where a full length paracervical electrode positioned in the posterior and lateral vaginal fornices and a single cutaneous electrode applied to the lower back area of the L5-S1 vertebral junction are attached to a single ISC.

FIG. 21 shows a side midline sagittal view of the electrical field created by the first mode of operation for the first embodiment of the present invention where a full length paracervical electrode positioned in the posterior and lateral vaginal fornices and a single cutaneous electrode applied to the lower back area of the L5-S1 vertebral junction are attached to a single ISC.

FIG. 22A shows, a side view illustration of a full length paracervical electrode accompanied by a left anterior partial-length paracervical electrodes and a left posterior partial-length paracervical electrode embedded in the covering of the left lateral portion of the proximal portion of the frame.

FIG. 22B shows a top view of a full length paracervical electrode accompanied by the right and left anterior partial-length paracervical electrodes embedded in the covering of the proximal portion of the frame.

FIG. 23 shows an illustration of the general location and shape of the electrical field produced by one of the ESC operating in the second mode of operation for the second embodiment of the present invention. The direct and targeted electrical neuromodulation of these nerves results in a reduction or elimination of perceived pelvic pain, as well as perceived external genital pain.

FIG. 24 shows a schematic representation of the shape of the two electrical fields created using the second mode of operation for the third embodiment of the present invention.

FIGS. 25A, 25B, 26A, 26B, 27A, 27B, 28A, 28B, 29A and 29B show, by way of examples, a collection of user interface screenshots that might be used to operate, control and modulate IVENS devices in accordance with an embodiment of the present invention.

### DETAILED DESCRIPTION OF EXEMPLARY EMBODIMENTS

Anatomical Terminology

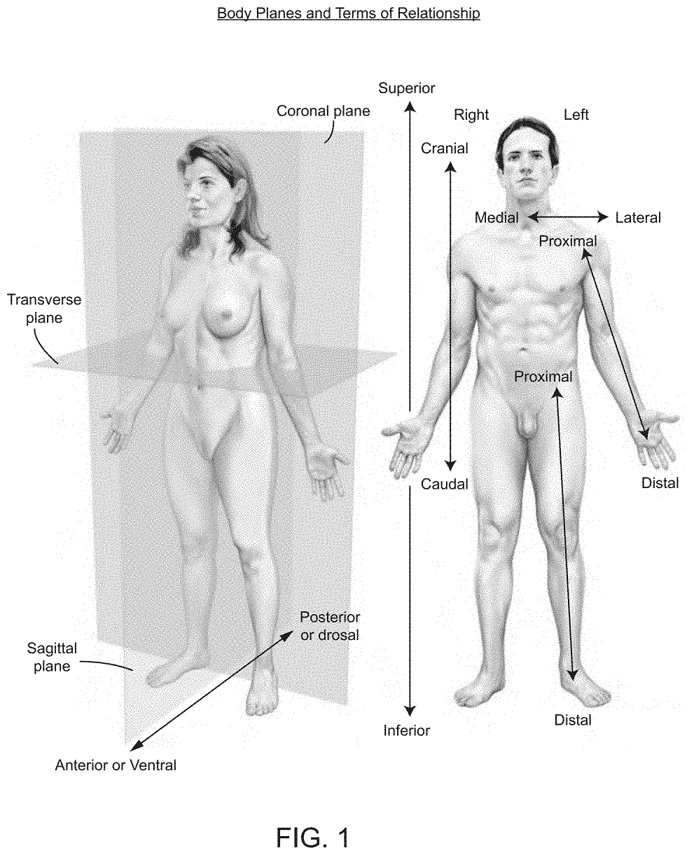

Turning now to the figures, FIG. 1 shows a diagram illustrating the conventional terms used to identify and describe the parts and planes of the human body, and the relationships therebetween. For purposes of this disclosure, “proximal” means nearer to the central portion of the body and distal means farther from the central portion of the body. The proximal portion of the vagina is the innermost and uppermost portion of the vagina near the uterine cervix. The distal portion of the vagina is the lowermost portion of the vagina near the vaginal orifice. Anterior means toward the front of the body and posterior means toward the back of the body. Medial means towards the vertical midline of the body, when viewed from the front or rear, and lateral means away from the vertical midline of the body, when viewed from the front or rear.

An anatomical plane (or anatomical section) refers to a view of anatomical structures in reference to a certain plane. For example, the median plane (or midline section) is the vertical plane that passes through the body longitudinally, front to back, dividing it into right and left halves. A sagittal plane is any vertical planes passing through the body that is parallel to the median plane. Sagittal planes divide the body into right and left parts. Therefore, the midline plane is a sagittal plane, but a sagittal plane need not be the midline plane. The coronal planes, also called the frontal planes, are vertical planes passing through the body, from one side to the opposite side, dividing the body into anterior (front) and posterior (back) portions. These vertical planes are at right angles)(90° to the median and sagittal planes.

Transverse planes are horizontal planes passing through the body, dividing it into superior (upper) and inferior (lower) parts. Transverse planes are at right angles)(90° to the median, sagittal and coronal planes. A superior view (sometimes called a “top view” or “bird's-eye” view) is a view that shows how an object would appear if when looking down on the object from a location above the object when the object is properly oriented within the body. An inferior view (sometimes called a “bottom view”) is a view that shows how an object would appear if looking up at the object from a location below the object when the object is properly orientated within the body.

Pelvic Anatomy

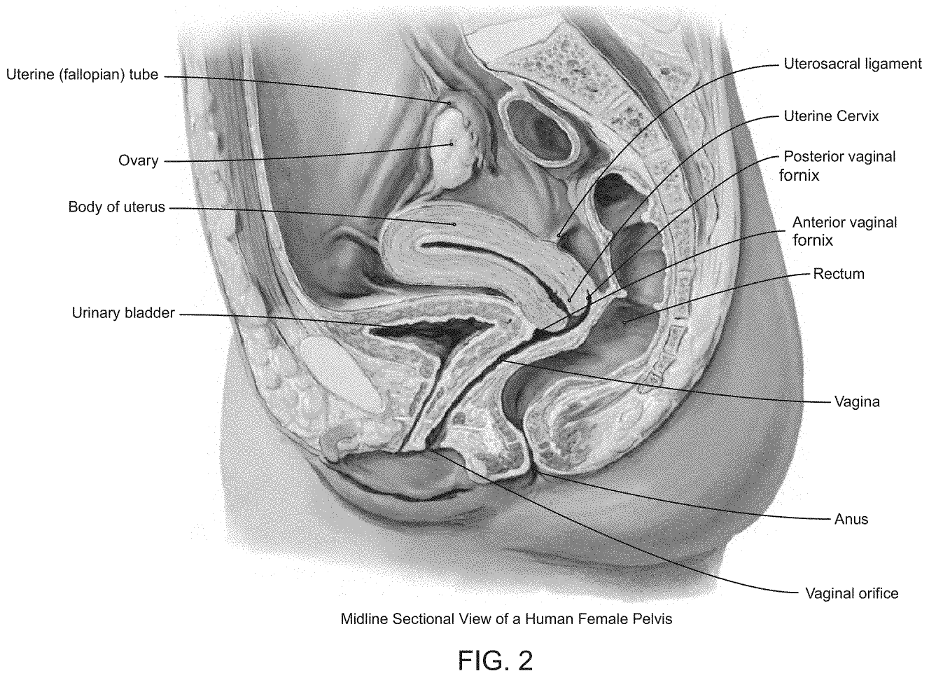

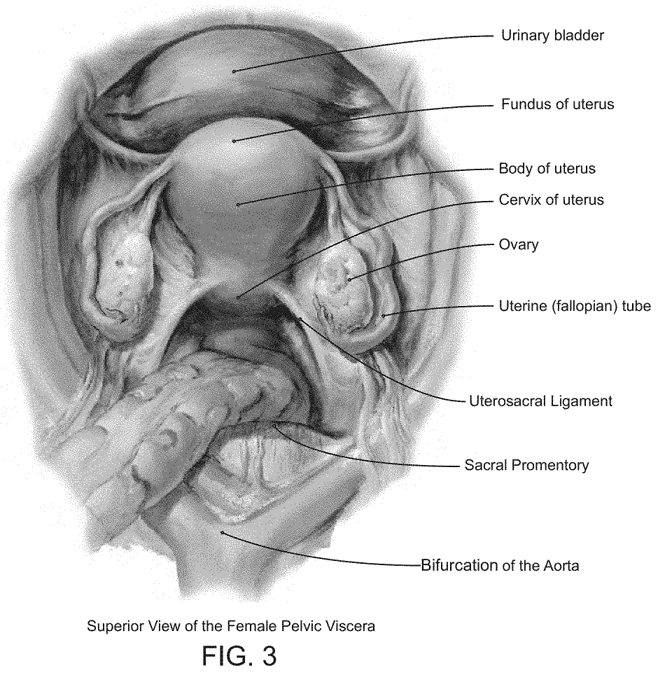

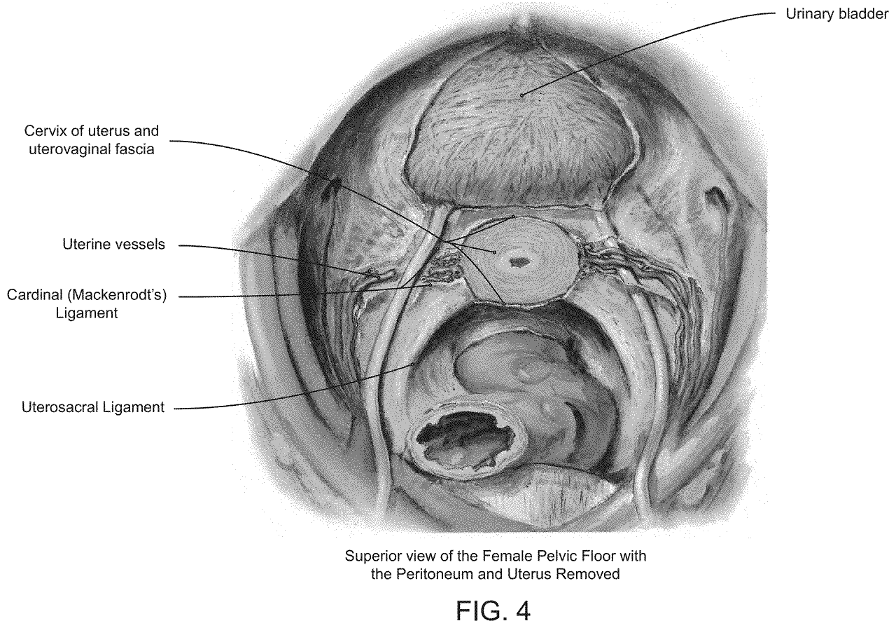

The pelvis is the lower part of the abdomen that is below the rim of the pelvic bones. FIG. 2 shows an illustration of a midline sectional view a human female pelvis. FIG. 3 shows an illustration of a superior view of the pelvic viscera, and FIG. 4 shows an illustration of a superior view of the female pelvic floor with the peritoneum and uterus removed. As shown in FIGS. 2, 3 and 4, the intraperitoneal female viscera that lie within the peritoneal cavity in the pelvis are the body of the uterus, fallopian tubes and ovaries. These organs are above, and supported by, the endopelvic fascia and ligaments that create the pelvic floor. The uppermost portion of the bladder lies above the pelvic floor but beneath the peritoneal lining of the pelvis. The subperitoneal female viscera that lie within or are below the pelvic floor include the uterine cervix, vagina, the lower portion of the bladder, the urethra and the lowermost part of the rectum.

Pelvic Neuroanatomy

Pelvic pain in women typically originate in their reproductive organs, bladder, pelvic floor or the peritoneal lining of the pelvis. The sensation of this pain is transmitted to the central nervous system through sympathetic, parasympathetic, and afferent sensory nerve fibers that traverse nerves and nerve plexuses in the abdomen and the pelvis, and the thoracic, lumbar, and sacral nerve roots.

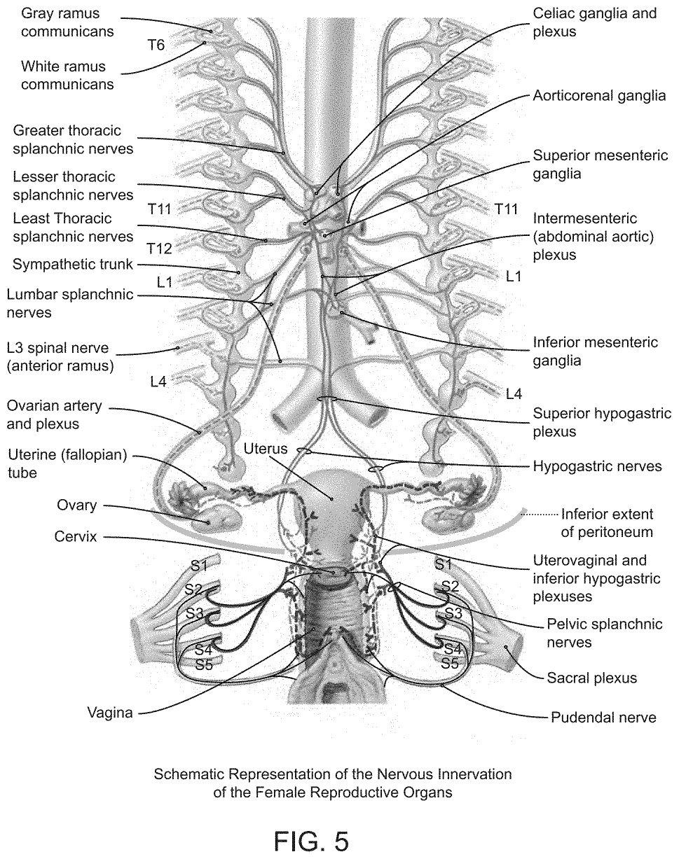

FIG. 5 shows a schematic representation of the nervous innervation of the female reproductive organs. As shown in FIG. 5, the sensation of pelvic pain from the intraperitoneal reproductive organs reaches the central nervous system by way of the pelvic nerves (as defied above) and the paracervical nerves (as defined above). The sensation of pelvic pain from the pelvic floor and the subperitoneal reproductive organs reaches the central nervous system by way of the pelvic nerves and the paracervical nerves. The sensation of pelvic pain from the lower vagina and the external genitalia reaches the central nervous system by way of the sacral nerves (as defined above).

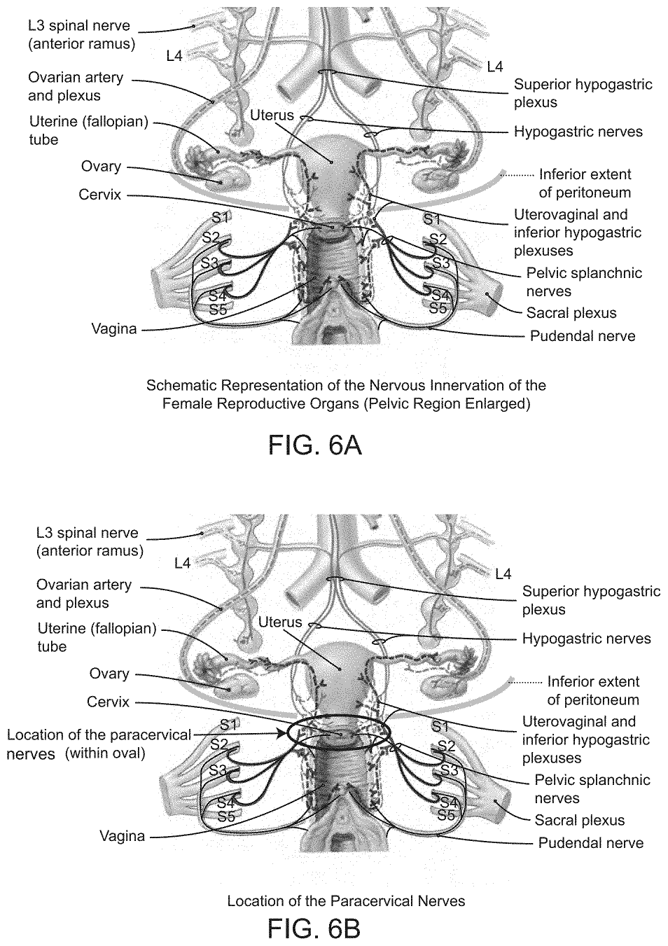

FIG. 6A is an enlarged view of the nervous intervention of the female reproductive organs. FIG. 6B shows the location of the paracervical nerves on each side of the cervix within the oval surrounding the cervix. This area represents a concentration of sympathetic, parasympathetic and afferent sensory nerve fibers in the area where the uterosacral ligaments and the cardinal ligaments attach to each side of the cervix. Additionally, these paracervical nerves are located just above (superior to and internal to) the area where the vaginal epithelium joins the cervix (the “paracervical vaginal epithelium”) in the posterior and lateral vaginal fornices.

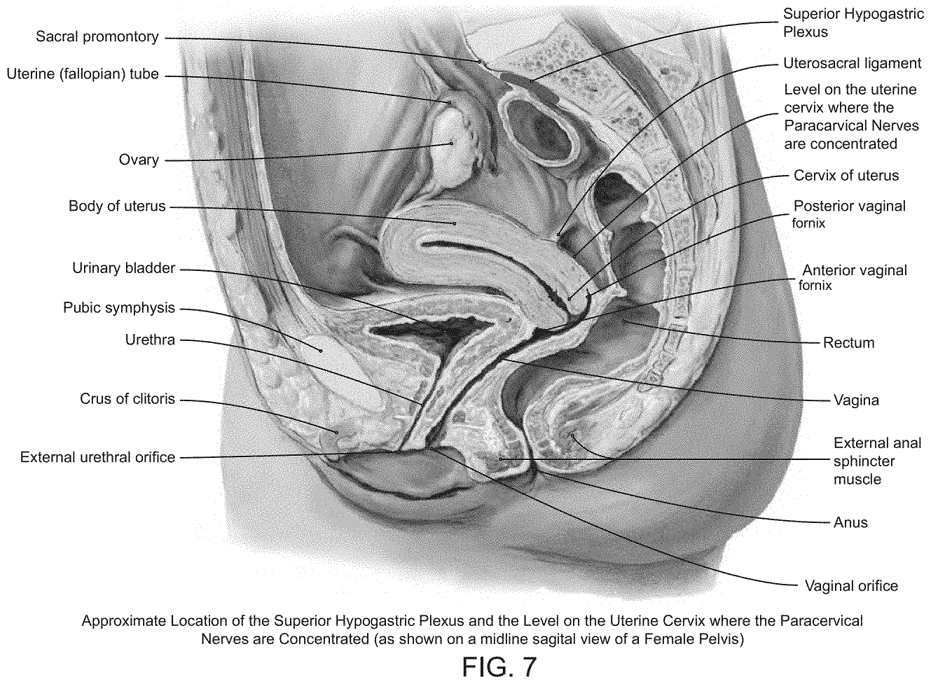

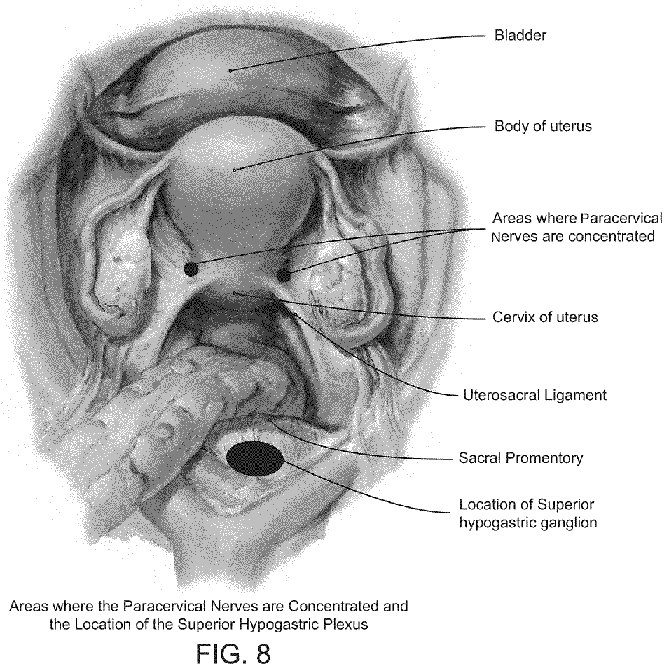

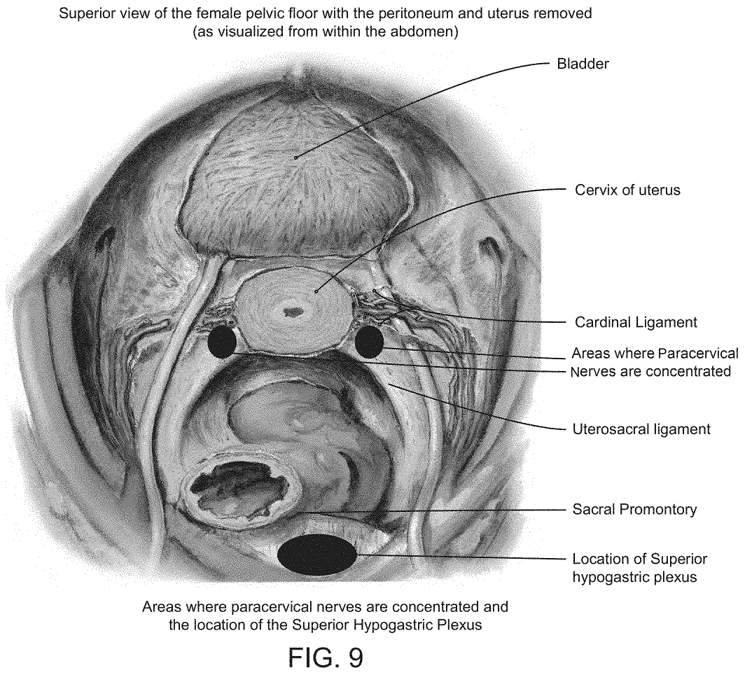

FIG. 7 is a midline sagittal view of a female pelvis that shows the approximate location of the superior hypogastric plexus, which lies over the bifurcation of the aorta in the presacral space anterior to the sacral promontory, and the level on the uterine cervix where the paracervical nerves are concentrated on each side of the cervix, lateral to the midline. FIG. 8 is a superior view of the pelvis viscera showing the areas where the of paracervical nerves are concentrated in the areas where the uterosacral and cardinal ligaments attach to each side of the cervix and the location of the superior hypogastric ganglion. FIG. 9 is a superior view of the pelvic floor with the uterus and perineum removed showing the areas where the of paracervical nerves are concentrated where the uterosacral and cardinal ligaments attach to each side of the cervix and the location of the superior hypogastric ganglion.

Intravaginal Electrical Neurostimulation Device for Treating Pelvic Pain and External Genital Pain

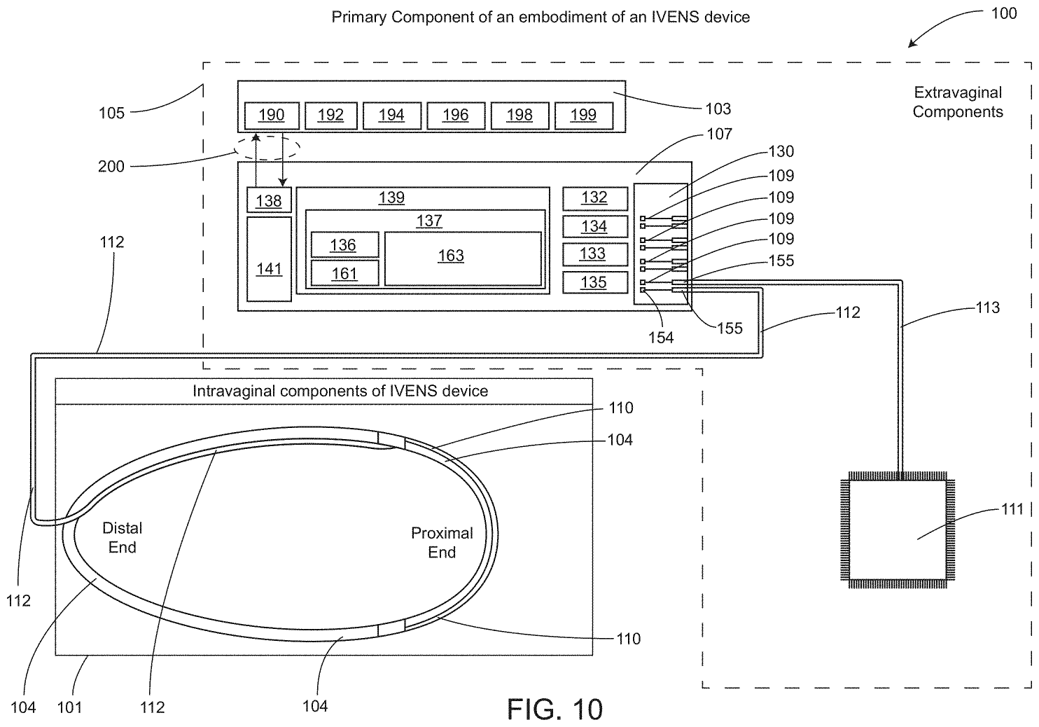

FIG. 10 is a schematic diagram illustrating in more detail the primary components of one embodiment of the IVENS device 100. As shown in FIG. 10, the primary components are a set of intravaginal components 101 and a set of extravaginal components 105. The set of intravaginal components 101 comprises a frame 104, a paracervical electrode (or electrodes) 110 and a paracervical electrode connecting wire (or wires) 112. The set of intravaginal components are designed and sized to be inserted into the patient's vagina. In some embodiments of the device, multiple partial-length paracervical electrodes 110 are embedded in the lateral portion of the covering of the proximal portion of the frame 104, along with the full length paracervical electrode 110. The set of extravaginal components 105 comprises the IVENS Stimulator (IS) 107, an optional external controller 103 and its related electrical components, a cutaneous electrode (or electrodes) 111, a cutaneous electrode connecting wire (or wires) 113, and the portion of the paracervical electrode connecting wire(s) 112 that extend to the outside of the vagina.

The Intravaginal Components of the IVENS Device

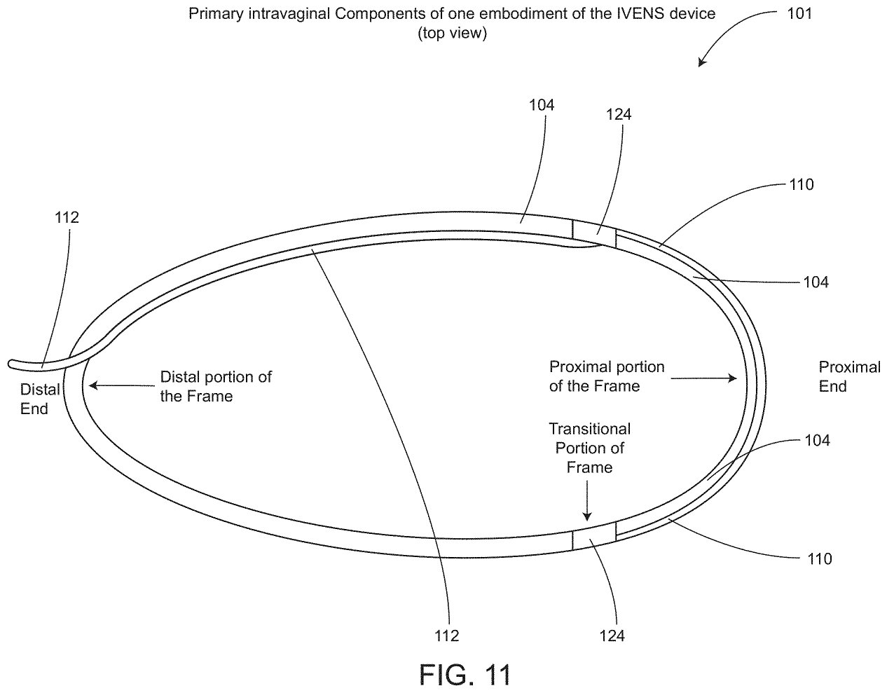

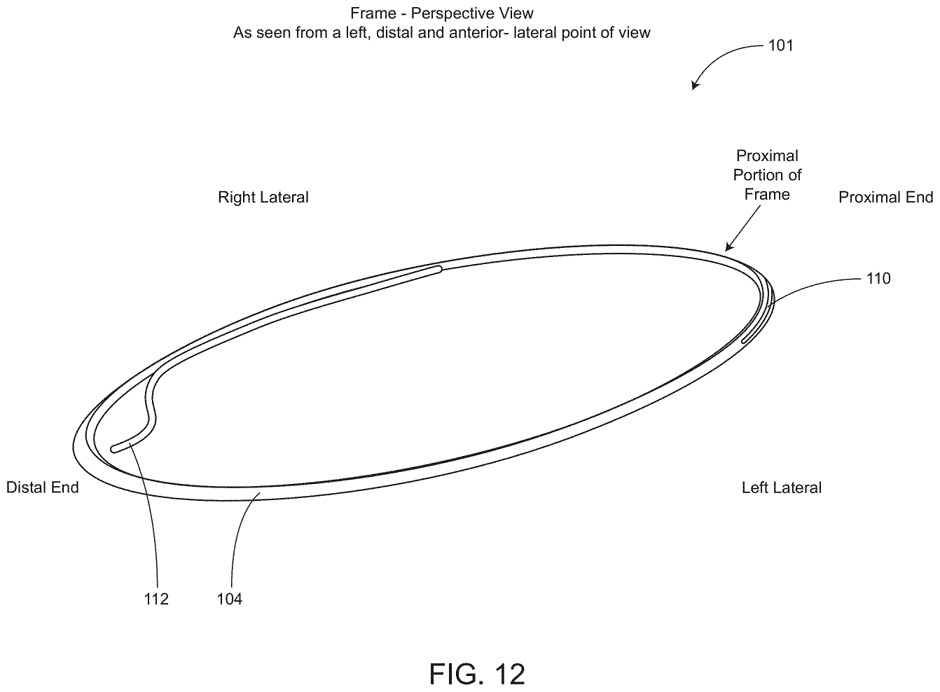

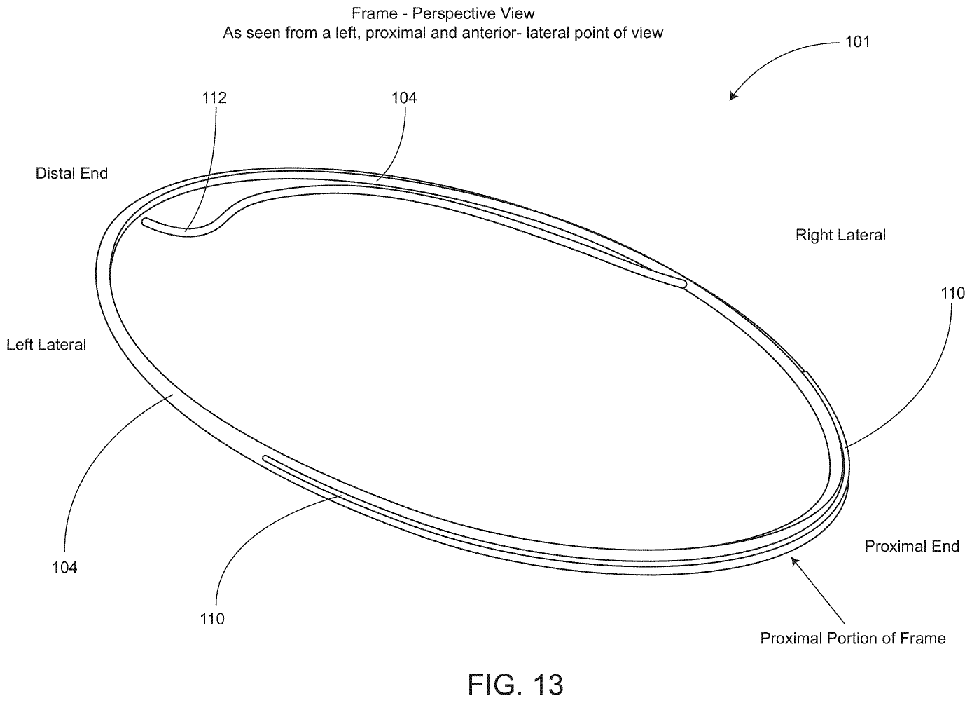

FIG. 11 shows a detailed top view of the primary elements of the set of intravaginal components 101 of one embodiment of the IVENS device 100. FIG. 12 shows a perspective view of the set of intravaginal components 101 of the IVENS device 100 as seen from a left, distal and anterior-lateral point of view. FIG. 13 shows a perspective view of the intravaginal components 101 of the IVENS device 100 as seen from a left, proximal and anterior-lateral point of view. It should be noted that, in FIGS. 10, 11, 12, and 13, only the single full length paracervical electrode 110 embedded in the distal portion of the frame 104 is illustrated for simplicity. In some embodiments of the device, multiple full-length paracervical electrodes and partial-length paracervical electrodes 110 may be embedded in the covering of the lateral portions of the proximal portion of the frame 104, either separately or alongside the first full length paracervical electrode 110.

The Frame

The frame 104 is a semi-elliptical structural element that is designed to be inserted and remain comfortably in a woman's vagina 146 when the IVENS device 100 is in use. Its core may be made of a semirigid, yet flexible material, such as a coiled spring made of metal or plastic or a rod made of metal, plastic or fiberglass or a combination of several of these materials. In addition, the material, or materials, used in the frame 104 may have different configurations and properties in different areas of the frame 104 to achieve the semi-rigidity or flexibility desired for that area of the frame 104. Notably, those skilled in the art will recognize and appreciate that a variety of alternative construction materials may be suitably substituted for the aforementioned materials without departing from the scope of the present invention.

In one embodiment of the IVENS device 100, the frame 104 has a posteriorly directed curvilinear shape when its lateral portions are compressed toward each other. The compressive forces are introduced, for example, when the woman squeezes the lateral portions of the frame 104 toward each other with the thumb and fingers of one hand prior to its insertion into the vagina 146. The frame 104 is constructed so that it has a propensity to return to its original shape when the compressive forces are reduced. The compressive forces are reduced after the device is inserted through the vaginal orifice and comes to rest completely within the vagina 146. Suitably, the proximal and distal ends of the frame 104 are typically the most flexible portions of the frame 104, allowing significant compression of the frame 104 along its longitudinal axis. Compression of the frame 104 along its longitudinal axis and the resulting posterior curvature of the frame 104 make insertion of the device into the vagina 146 easier to accomplish. Once the frame 104 is completely inserted into the vagina 146 and released, the frame 104 returns to its original configuration where the distal end of the frame 104 rests upon the anterior vaginal wall 141 behind the synthesis pubis 142 and the proximal portion of the frame 104 rests upon the vaginal epithelium in the posterior vaginal fornix 150. In this configuration inside the vagina 146, the paracervical electrode 110 embedded in the surface of the covering of the proximal portion of the frame 104 will come into contact with the vaginal epithelium in the lateral and posterior vaginal fornices. The lateral portions of the frame 104 are configured to gently press against the lateral walls of the vagina 146, keeping the frame 104 in the proper position within the vagina 146. The core elements of the frame 104 may be covered with a smooth, flexible medical grade covering, such as silicone rubber.

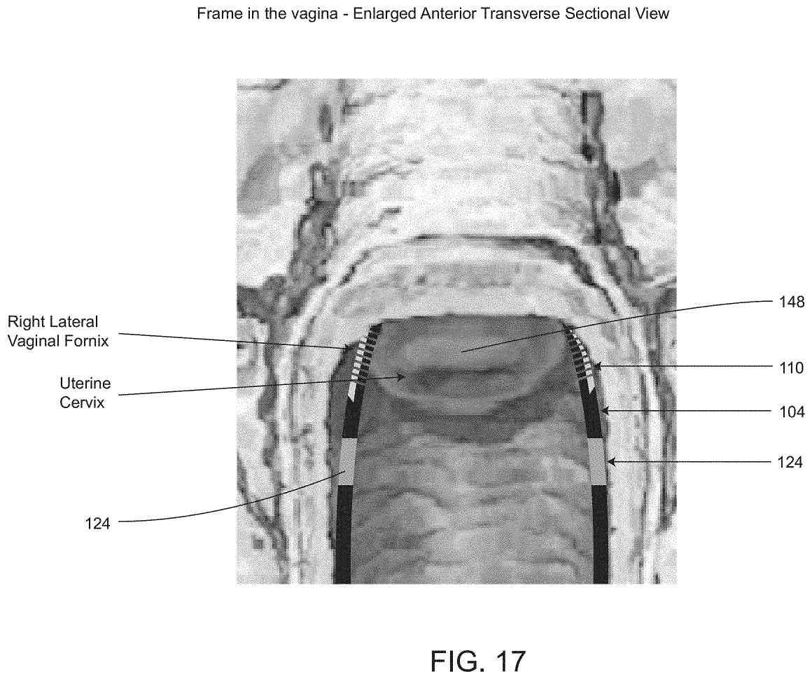

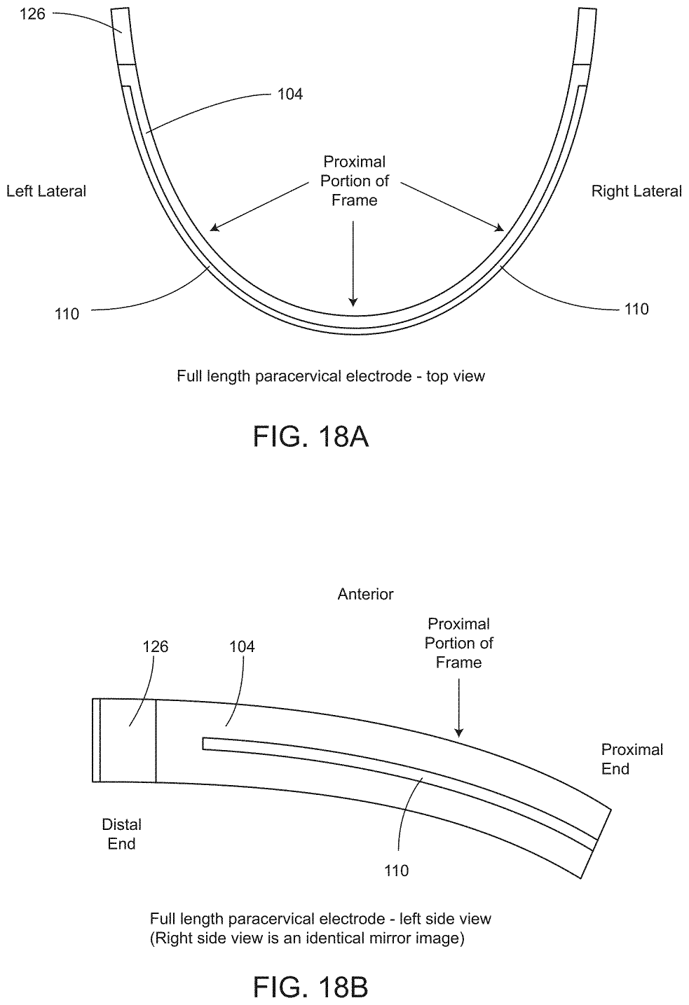

In another embodiment of the present invention, a “transitional” portion 124 may be placed in both of the lateral portions of the frame 104 at a point approximately seventy-five percent of the distance from the distal end to the proximal end of the frame 104. See FIGS. 17, 18A and 18B. The transitional portion 124 of the frame 104 may be curved slightly posteriorly and be more flexible than the other portions of the frame 104 to facilitate the positioning of the proximal portion of the frame 104 in the posterior and lateral vaginal fornices when the device is introduced into the vagina 146. The propensity of the transitional portion 124 of the frame 104 to return to its original configuration once any pressure applied to it is released will cause the proximal portion of the frame 104 to apply gentle pressure superiorly and posteriorly to the vaginal epithelium in the posterior and lateral vaginally fornices, keeping the paracervical electrodes 110 in contact with the paracervical vaginal epithelium in the lateral fornices and properly positioned to deliver electrical stimulation to the paracervical nerves.

Embodiments of the present invention may be manufactured in a variety of different sizes, and have a variety of longitudinal lengths for the frame 104 (for example from 6.5 to 8.5 centimeters, in increments of 5 millimeters) in order to accommodate the various vaginal depths of the women who may use the device. The lateral flexibility of the frame 104 increases the device's ability to address and accommodate differences in vaginal caliber among different women. The increased flexibility of the transitional portion 124 of the frame 104 and its propensity to return to its original configuration, when pressure placed upon it is released helps maintain the proper positioning of the paracervical electrodes 110 in the lateral vaginal fornices.

In an embodiment of the device intended for the treatment of postsurgical pain following a hysterectomy, the shape of the proximal portion of the frame 104 will be modified so it can rest in the upper the vagina 146 when the uterine cervix is absent.

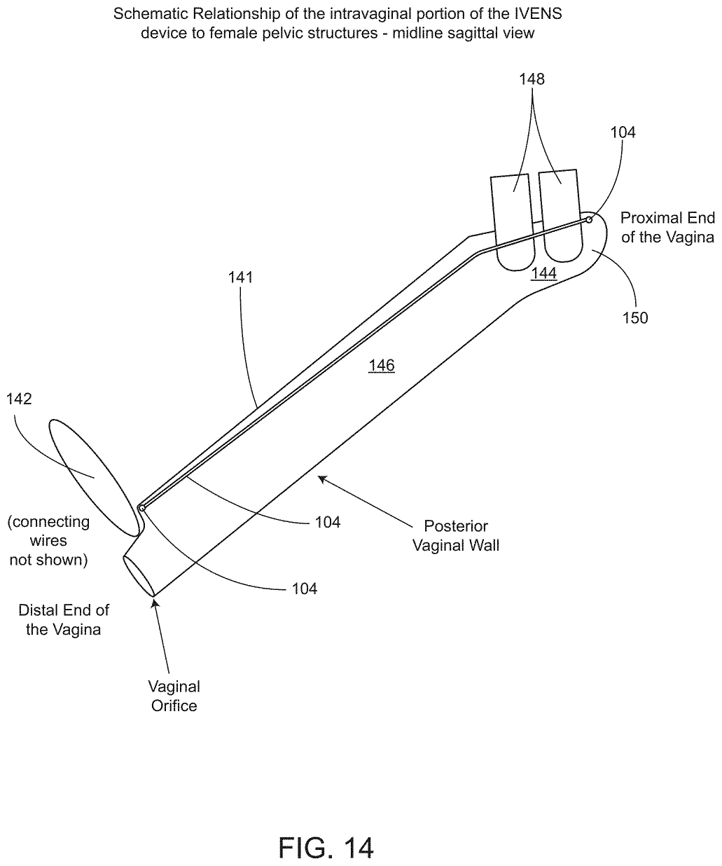

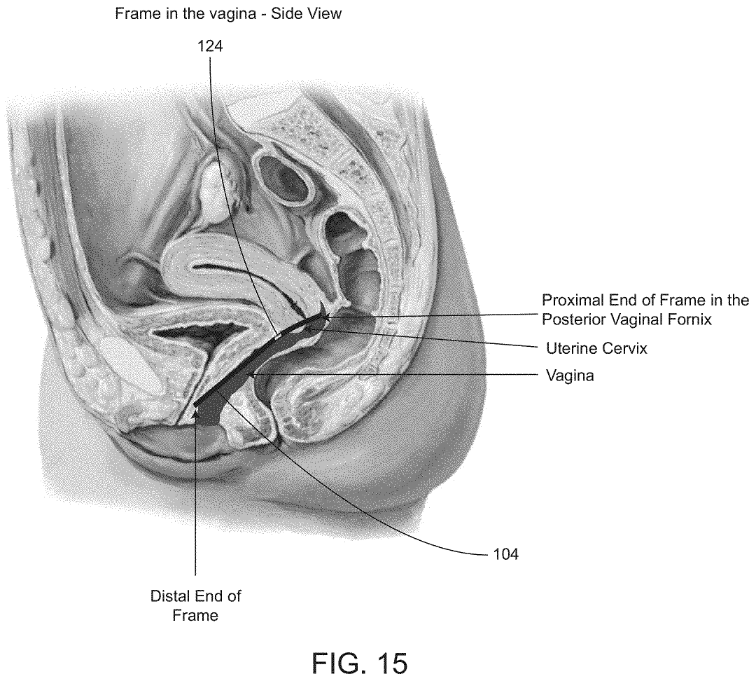

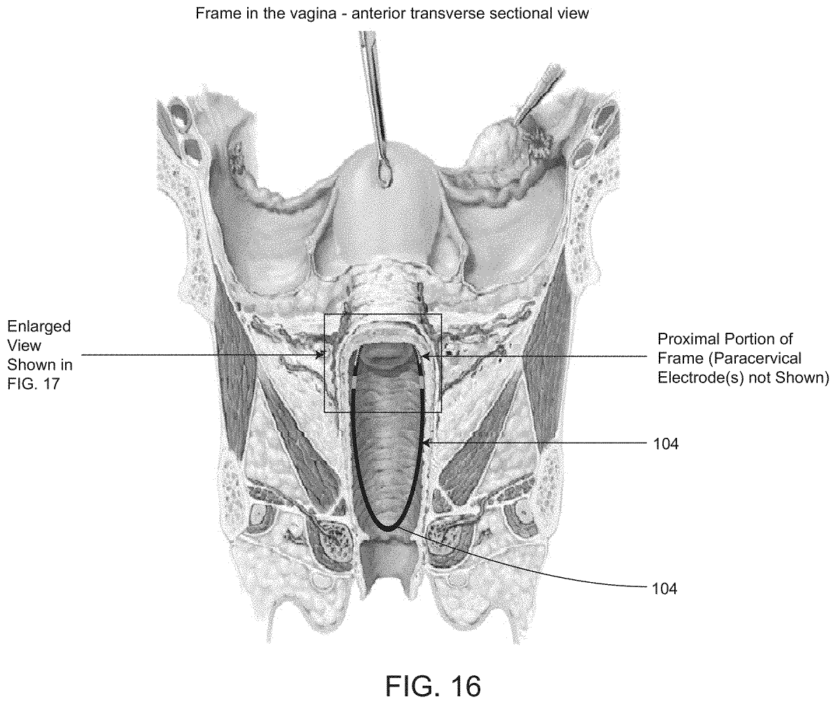

FIGS. 14, 15, 16 and 17 show schematic diagrams illustrating the typical placement and orientation of the frame 104 within the vagina in accordance with some embodiments of the present invention. As shown in FIGS. 14, 15, 16 and 17, the distal end of the frame 104 rests against the anterior vaginal wall behind the symphysis pubis. The proximal end of the frame 104 rests against the vaginal epithelium in the posterior vaginal fornix. The proximal portion of the frame 104 and the paracervical electrodes 110 residing thereon rests in the posterior and lateral vaginal fornices. The lateral portions of the frame 104 press gently on the lateral walls of the vagina 146 to help keep the device in the proper position within the vagina 146. FIG. 17 shows an enlarged view of the upper vagina 146 and uterine cervix 148 with the proximal end of the frame 104 in-situ to illustrate the proper positioning of the paracervical electrodes 110 in the posterior and lateral vaginal fornices. In FIG. 17, the dashed portions of paracervical electrode(s) 110 and the dashed portions of the frame 104 reside lateral to and posterior to the uterine cervix 148.

The Paracervical Electrodes

A paracervical electrode 110 comprises one or more wires, capable of carrying or conducting electrical current, which are embedded in the covering of the proximal portion of the frame 104. In one embodiment, the surface area of a paracervical electrode 110 may be increased by attaching a thin “wafer” of electrode material (that might be round, square or rectangular in cross section, for example) to the paracervical electrode (or electrodes) 110 embedded in the covering of the proximal portion of the frame 104. The wire(s) and wafers comprising the paracervical electrode 110 are either not insulated, or minimally insulated, so the electrical stimulation generated by the electrical stimulation generator and transmitted to the paracervical electrodes 110 is delivered to the paracervical vaginally epithelium, and hence, the pelvic nerves, paracervical nerves and sacral nerves.

For the purposes of this disclosure, three embodiments of the IVENS device 100 are presented.

In the first embodiment of the present invention, the paracervical electrode 110 is a full length paracervical electrode 110. The full length paracervical electrode 110 extends from the junction of the transitional portion 124 of the frame 104 and the proximal portion of frame 104 on one side of the frame 104 to the same position on the other side of the frame 104. When the frame 104 is properly positioned in the vagina 146, the full length paracervical electrode 110 will be in contact with the vaginal epithelium of both lateral vaginal fornices and the posterior vaginal fornix.

In the second embodiment of the present invention, two partial length paracervical electrodes 110 are embedded in the covering of the distal and lateral portion of the proximal portion of the frame 104 with one partial-length paracervical electrode 110 present on each side of the frame 104. When the frame 104 is properly positioned in the vagina 146, the two partial-length paracervical electrodes 110 will be in contact with the vaginally epithelium in the lateral vaginal fornices on each side of the cervix 148.

In the third embodiment of the present invention four partial-length paracervical electrodes 110 are embedded in the covering of the distal and lateral portion of the proximal portion of the frame 104 with a pair of partial-length paracervical electrodes 110 present on each side of the frame 104. When the frame 104 is properly positioned in the vagina 146, the two pairs of partial-length paracervical electrodes 110 will be in contact with the vaginal epithelium in the lateral vaginal fornices, with one pair of paracervical electrodes 110 on each side of the uterine cervix 148.

Those skilled in the art should recognize that other embodiments can be created using multiple paracervical electrodes 110 embedded in the distal portion of the frame 104 and multiple cutaneous electrodes 111 applied to the lower back, or other areas of the lower abdomen or pelvis which can be paired together to create a wide variety of electrical stimulation circuits.

The Paracervical Electrode Connecting Wires

The full length paracervical electrode 110 and each partial-length paracervical electrode 110 embedded in the covering of the proximal portion of the frame 104 are attached to individual paracervical electrode 110 connecting wires that have color-coded electrical connections at their terminus that will attached to one of the two electrical connections in an ISC 109 port on the IS 107.

The color-coding of the terminal ends of the paracervical electrode 110 connecting wires and the cutaneous electrode connecting wires 113 (as described below) will allow the patient to use a color-coded chart to connect different combinations of electrodes to multiple ISCs 109 to create multiple electrical stimulation circuits to enable creating multiple electrical fields at the same time.

FIGS. 18A and 18B show a top view and a side view illustration of a full length paracervical electrode 110 embedded in the covering of the proximal portion of the frame 104 respectively.

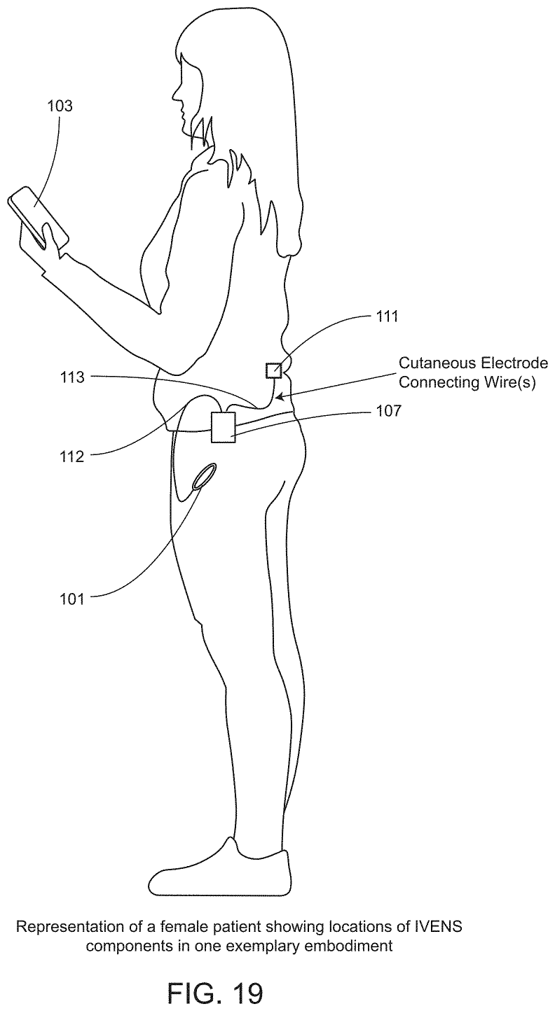

FIG. 19 shows a representation of a female patient wearing an IVENS device 100 showing the approximate location of the intravaginal components 101 and extravaginal components 105 of the IVENS device 100 required by the different embodiments of the device presented herein. As shown in FIG. 19 the set of intravaginal components, which is positioned in the patient's upper vagina, is connected by a paracervical electrode connecting wire 112 to the set of extravaginal components 105, which includes an IVENS stimulator 107 (which contains the electrical stimulation generator 130), a cutaneous electrode 111 (positioned on the patient's lower back) and a cutaneous electrode connecting wire 113, which electrically couples the cutaneous electrode 111 to the IS 107. In some embodiments, the extravaginal components 105 may also include a handheld external controller 103, which may be installed on, or integrated into, a smartphone or tablet computer. Notably, the placement of a single cutaneous electrode 111 on the patient's lower back is required for the first mode of operation for the first embodiment of the present invention presented herein, and the placement of two cutaneous electrodes 111 on the patient's lower back are required for the first mode of operation for the second embodiment of the present invention presented herein. Cutaneous electrodes are not required for the third embodiment of the present invention presented herein.

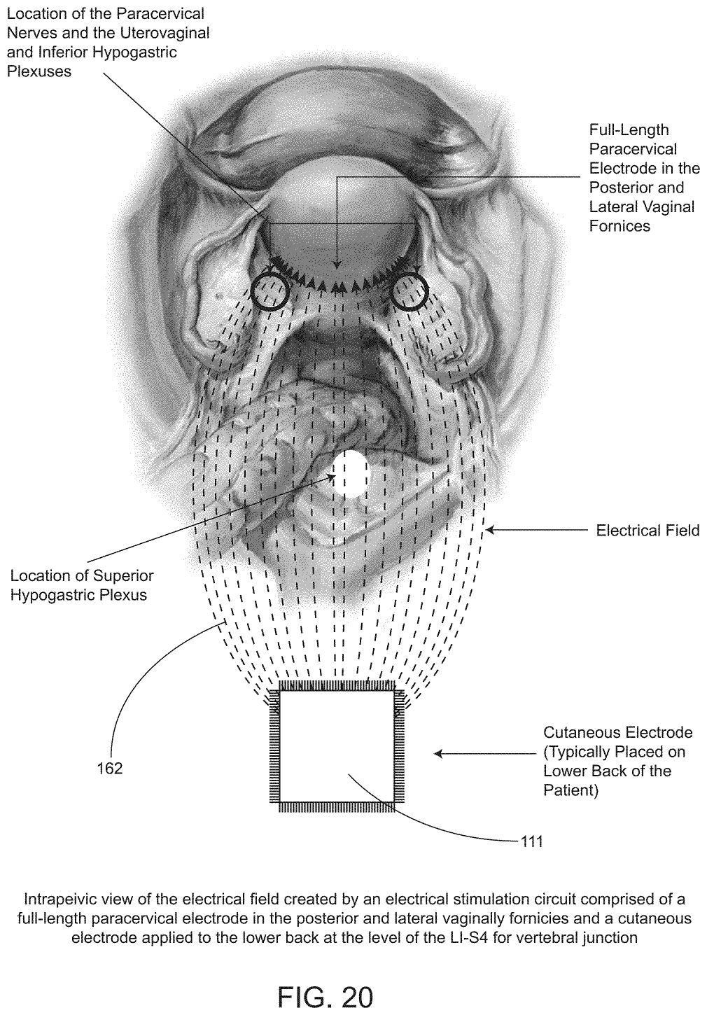

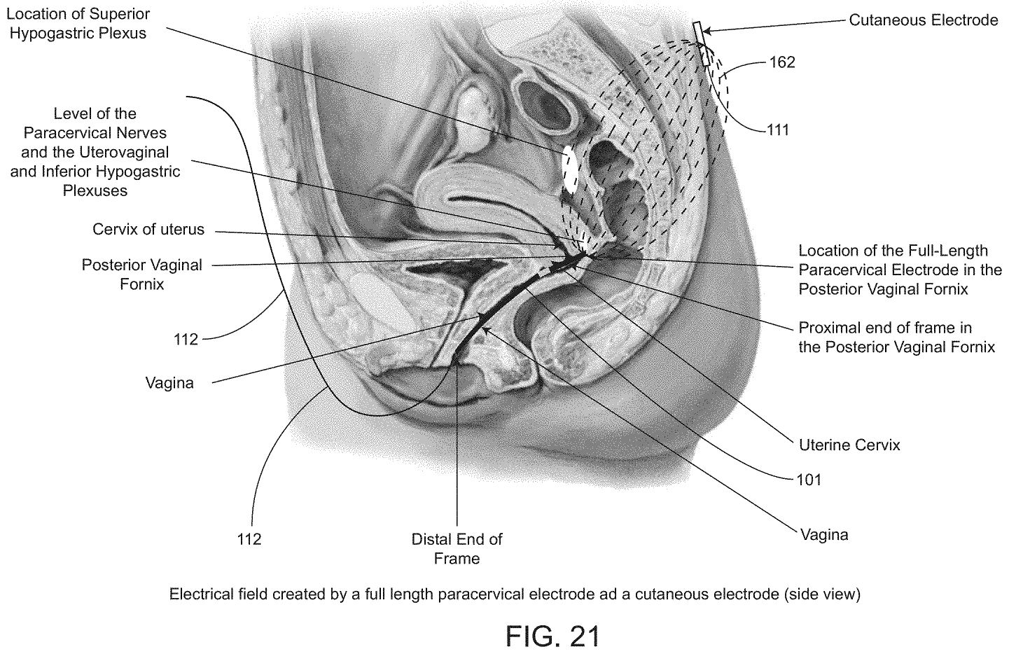

FIG. 20 shows an intrapelvic view of the electrical field 162 created by the first mode of operation for the first embodiment of the present invention, wherein a full length paracervical electrode 110 positioned in the posterior and lateral vaginal fornices and a single cutaneous electrode 111 applied to the lower back area of the L5-S1 vertebral junction are attached to a single ISC 109. FIG. 21 shows a side midline sagittal view of the electrical field 162 created by the first mode of operation for the first embodiment of the present invention where a full length paracervical electrode 110 positioned in the posterior and lateral vaginal fornices and a single cutaneous electrode 111 applied to the lower back area of the L5 — 51 vertebral junction are attached to a single ISC 109. As shown in FIGS. 20 and 21, in this first mode of operation for the first embodiment of the present invention, the IVENS device 100 creates an electrical field 162 between the full length paracervical electrode 110 positioned in the posterior and lateral vaginal fornices of the upper vagina and the cutaneous electrode 111 placed on the skin of the lower back in the area of the L5-S1 vertebral junction. Thus, the electronic signals sent to the ESG 130 in the IS 107 by the microprocessor operating under the control of the IDCA 137 in the static memory causes the electrical stimulation generator to transmit an electrical stimulation patterns through an ISC 109 to create an electrical stimulation circuit between the full length paracervical electrode 110 positioned in the posterior and lateral vaginal fornices and a cutaneous electrode 111 positioned externally on the lower back in the area of the L5-S1 vertebral junction to create an electrical field that will influence the pelvic nerves, the paracervical nerves and the sacral nerves to provide direct and targeted electrical neuromodulation of these nerves to reduce or eliminate pelvic pain and external genital pain.

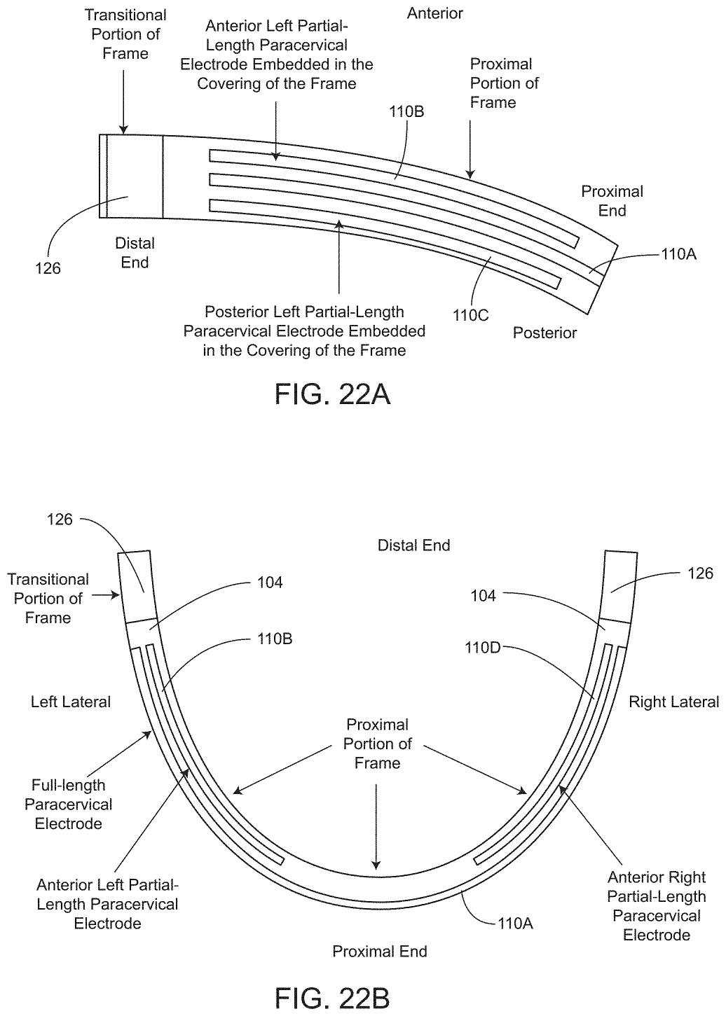

FIG. 22A shows, a side view illustration of a full length paracervical electrode 110A accompanied by a left anterior partial-length paracervical electrode 110B and a left posterior partial-length paracervical electrode 110C embedded in the covering of the left lateral portion of the proximal portion of the frame 104. A side view illustration of a full length paracervical electrode 110 accompanied by a right anterior partial-length paracervical electrode 110 and a right posterior partial-length paracervical electrode 110 embedded in the covering of the right lateral portion of the proximal portion of the frame 104 would be a mirror image of FIG. 22A. FIG. 22B shows a top view of a full length paracervical electrode 110A accompanied by the left and right anterior partial-length paracervical electrodes 110B and 110D, respectively, embedded in the covering of the proximal portion of the frame 104.

The ability of the ESG 130 to create and deliver multiple ESPs to multiple ISCs 109 simultaneously allows for the creation of a variety of similar and dissimilar electrical fields between multiple pairs of electrodes appropriately placed in the proximity of the pelvic nerves, paracervical nerves, and sacral nerves at the same time allows multiple modes of operation for multiple embodiments of the present invention. Thus, the electronic signals sent to the ESG 130 in the IS 107 by the microprocessor operating under the control of the IDCA 137 in the static memory causes the electrical stimulation generator to transmit multiple electrical stimulation patterns through multiple ISCs 109 to create multiple electrical stimulation circuits to create multiple electrical fields between multiple pairs of electrodes that are placed in specific locations within or around the pelvis to specifically target the pelvic nerves, the paracervical nerves and the sacral nerves causing electrical neuromodulation of these nerves to reduce or eliminate pelvic pain and external genital pain.

For the purpose of further describing and illustrating exemplary embodiments of the present invention, three embodiments of the IVENS device 100 will now be described in some detail.

In the first embodiment, the paracervical electrode is a full-length paracervical electrode 110A.

In the second embodiment of the present invention, two partial length paracervical electrodes 110B and 110D. In this embodiment, a first partial-length paracervical electrode 110B is embedded in the covering on the right side of the distal and lateral portions the frame 104, and a second partial-length paracervical electrode 110D is embedded in the covering on the left side of the distal and lateral portion of the frame 104. When the IVENS device 100 is properly positioned in the vagina, the first partial-length paracervical electrode 110B will be in contact with the vaginal epithelium in the right lateral vaginal fornix and the second partial-length paracervical electrode 110D will be in contact with the vaginal epithelium in the left lateral vaginal fornix.

In the third embodiment of the present invention (not shown), four partial-length paracervical electrodes are present, with two partial-length paracervical electrodes embedded in the covering of the distal and lateral portion of the proximal portion of the frame 104 on each side of the frame 104.

Persons skilled in the art will recognize and appreciate, however, that other embodiments can be created using multiple paracervical electrodes 110 embedded in the proximal portion of the frame 104 and multiple cutaneous electrodes 111 applied to the lower back, or other areas of the lower abdomen or pelvis, which can be paired together to create a wide variety of electrical stimulation circuits and a wide variety of overlapping electrical fields.

While the first and second embodiments require at least one cutaneous electrode 111, the third embodiment does not necessarily require using a cutaneous electrode 111.

The first exemplary embodiment has at least one mode of operation. The second embodiment has at least two modes of operation. And the third embodiment has at least two modes of operation. Therefore, when considered together, the three exemplary embodiments may be operated in a total of five different modes of operation. These five different modes of operation for the IVENS device 100 will now be described in more detail with reference to FIGS. 20, 21, 23 and 24, as well as Table 1 below, captioned “Table 1—Exemplary Embodiments and Modes of Operation of the IVENS device 100.” These modes of operation use a variety of different combinations of electrode types, electrode placement locations, and one or two ISCs 109. However, persons skilled in the art will recognize and appreciate the fact that additional modes of operation can be created using additional combinations of electrode types, electrode placement locations, and additional ISCs 109.

The electrode types, electrode placement locations, and number of ISCs 109. used in the embodiments and the modes of operation presented hereinafter are summarized in the table below. In this table, the labels in the first column identify the embodiments and modes of operations as follows:

E1M1=Embodiment 1—Mode of Operation 1,

E2M1=Embodiment 2—Mode of Operation 1,

E2M2=Embodiment 2—Mode of Operation 2,

E3M1=Embodiment 3—Mode of Operation 1, and

E3M2=Embodiment 3—Mode of Operation 2.

TABLE 1
Exemplary Embodiments and Modes of Operation of the IVENS Device

| | IS Channel | Types of Electrodes Used | Location or Position of Electrodes |
|---|---|---|---|
| E1M1 | 1st ISC | A full length paracervical electrode | Upper vagina - posterior and lateral vaginal fornicies |
| | | A cutainous electrode | Lower back near the level of the L5-S1 vertebral junction |
| E2M1 | 1st ISC | | Upper vagina - right lateral vaginal fornix |
| | | A first cutainous electrode | Lower back to the right of the midline near the level |
| | | | of the L5-S1 vertebral junction |
| | 2nd ISC | A second partial-legnth paracervical electrode | Upper vagina - left lateral vaginal fornix |
| | | A second cutainous electrode | Lower back to the left of the midline near the level |
| | | | of the L5-S1 vertebral junction |
| E2M2 | 1st ISC | A first partial-legnth paracervical electrode | Upper vagina- right lateral vaginal fornix |
| | | A second partial-legnth paracervical electrode | Upper vagina - left lateral vaginal fornix |
| E3M1 | 1st ISC | A first partial-legnth paracervical electrode | Upper vagina - right anterior lateral vaginal fornix |
| | | A second partial-legnth paracervical electrode | Upper vagina - left anterior lateral vaginal fornix |
| | 2nd ISC | A third partial-legnth paracervical electrode | Upper vagina - right posterior lateral vaginal fornix |
| | | A fourth partial-legnth paracervical electrode | Upper vagina - left posterior lateral vaginal fornix |
| E3M2 | 1st ISC | A first partial-legnth paracervical electrode | Upper vagina- right anterior lateral vaginal fornix |
| | | A second partial-legnth paracervical electrode | Upper vagina - right posterior lateral vaginal fornix |
| | 2nd ISC | A third partial-legnth paracervical electrode | Upper vagina - left anterior lateral vaginal fornix |
| | | A fourth partial-legnth paracervical electrode | Upper vagina - left posterior lateral vaginal fornix |

As shown in Table 1, in a first mode of operation for the first embodiment of the present invention (see the rows associated with the label E1M1 in Table 1), a full length paracervical electrode 110 is positioned in the posterior and lateral vaginal fornices of the upper vagina and a single cutaneous electrode 111 is attached to the lower back in the area of the L5-S1 vertebral junction. For this embodiment, the full length paracervical electrode and the cutaneous electrode 111 are attached by connecting wires to a single ISC 109 of the electrical stimulation generator 130 in the IS 107. The ESG 130 is configured to generate an electrical field between the full length paracervical electrode and the cutaneous electrode 111 using the single ISC 109. This electric field will influence and neuromodulate the pelvic nerves, the paracervical nerves and the sacral nerves. The direct and targeted electrical neuromodulation of these nerves results in a reduction or elimination of perceived pelvic pain, as well as perceived external genital pain. This first embodiment of the present invention was discussed above in connection with the descriptions of FIGS. 20 and 21, which show, relative to the pelvis, the general location and shape of the electrical field created by the ESC while the device is operating in this first mode of operation for the first embodiment of the present invention.

In the second embodiment of the present invention (see the rows associated with the labels E2M1 and E2M2 in Table 1), a first partial-length paracervical electrode on the right side of the frame 104 is positioned in the right lateral vaginal fornix, and a second partial-length paracervical electrode on the left side of the frame 104 is positioned in the left lateral vaginal fornix. In a first mode of operation for this second embodiment of the present invention, the first partial length paracervical electrode 110 (positioned in the right lateral vaginal fornix) and a first cutaneous electrode 111 (placed on the skin of the lower back in the area of the L5-S1 vertebral junction to the right of the midline) are attached to the first ISC 109 on the IS 107. In addition, a second partial length paracervical electrode 110 (positioned in the left lateral vaginal fornix) and a second cutaneous electrode 111 (placed on the skin of the lower back in the area of the L5-S1 vertebral junction to the left of the midline) are attached to a second ISC 109 on the IS 107.

In a first mode of operation for the second embodiment (E2M1) of the present invention, the electrical stimulation generator is configured to generate a first electrical field located between the first partial-length paracervical electrode and the first cutaneous electrode 111 using the first ISC 109, and a second electrical field between the second partial-length paracervical electrode 110 and the second cutaneous electrode 111 using the second ISC 109. FIGS. 20 and 21 also show the general location and shape of one of the two electrical fields created by each of the ESC's while operating in this first mode of operation for the second embodiment of the present invention. In some situations, the first and second electrical fields may have identical electrical stimulation patterns. In other situations, the first and second electrical fields may have different electrical stimulation patterns. The two electrical fields may exist simultaneously, consecutively or in an alternating pattern. Each of these electrical fields are targeted to influence and neuromodulate the pelvic nerves, the paracervical nerves and the sacral nerves. The direct and targeted electrical neuromodulation of these nerves results in a reduction or elimination of perceived pelvic pain, as well as perceived external genital pain.

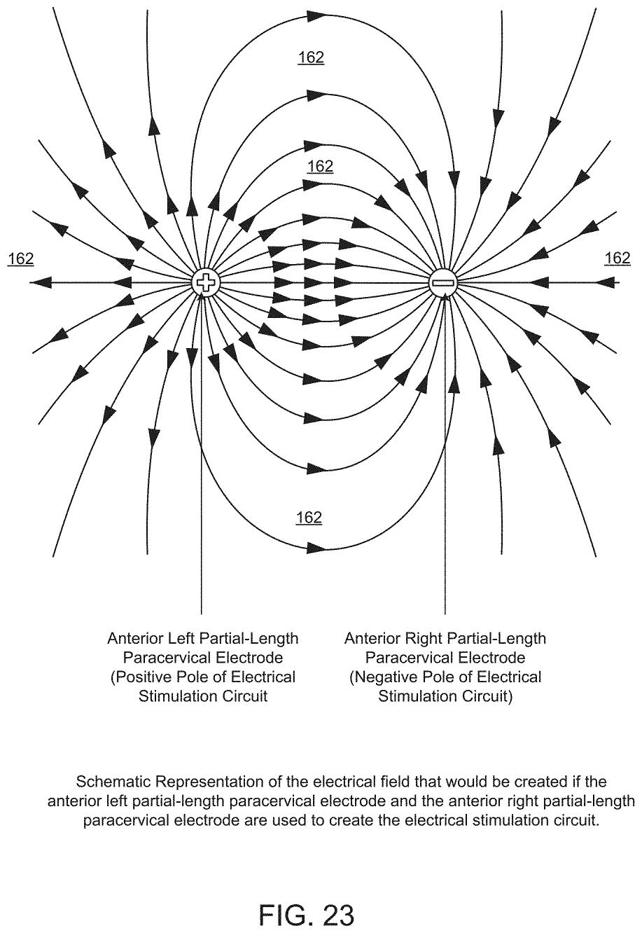

In a second mode of operation for this second embodiment of the present invention (see the rows associated with the E2M2 label in Table 1 above), a first partial-length paracervical electrode 110 on the right side of the frame 104 is positioned in the right lateral vaginal fornix and a second partial-length paracervical electrode 110 on the left side of the frame 104 is positioned in the left lateral vaginal fornix and both electrodes are attached to a single ISC 109 (the first ISC) on the ESG 130 of the IS 107. The electrical pulse generator is configured to generate a single electrical field between the first partial-length paracervical electrode 110 and the second partial-length paracervical electrode 110, which provides targeted electrical neuromodulation of the paracervical nerves and, to a lesser extent, additional neuromodulation of the pelvic and sacral nerves. The direct and targeted neuromodulation of the paracervical nerves, pelvic nerves and sacral nerves in this fashion results in a reduction or elimination of perceived pelvic pain, as well as perceived pain from the perineum and external genitalia. FIG. 23 shows an illustration of the general location and shape of the electrical field produced by one of the ESC operating in the second mode of operation for the second embodiment of the present invention. The direct and targeted electrical neuromodulation of these nerves results in a reduction or elimination of perceived pelvic pain, as well as perceived external genital pain.

In a third embodiment of the present invention (identified with the labels E3M1 and E3M2 of Table 1 above), four partial-length paracervical electrodes 110 are present. Two of the four partial-length paracervical electrodes 110 are embedded in the covering of the distal and lateral portion of the proximal portion of the frame 104 on each side of the frame 104. When the frame 104 is properly positioned in the vagina, the first partial-length paracervical electrode 110 is positioned in the right anterior Lateral vaginal fornix, the second partial-length paracervical electrode 110 is positioned in the left anterior lateral vaginal fornix, the third partial-length paracervical electrode 110 is positioned in the right posterior lateral vaginal fornix, and the fourth partial-length paracervical electrode 110 is positioned in the left posterior lateral vaginal fornix.

In a first mode of operation for this third embodiment (see the rows associated with the E3M1 label in Table 1), the first partial-length paracervical electrode 110 (positioned in the right anterior lateral vaginal fornix) and the second partial-length paracervical electrode 110 (positioned in the left anterior lateral vaginal fornix) are attached to the first ISC 109 of the ESG 130 in the IS 107, and the third partial-length paracervical electrode 110 (positioned in the right posterior lateral vaginal fornix) and the fourth partial-length paracervical electrode 110 (positioned in the left posterior lateral vaginal fornix) are attached to the second ISC 109 of the ESG 130 in the IS 107. The ESG 130 is configured to generate an electrical field between the first partial-length paracervical and the second partial-length electrodes using the first ISC 109, and a second electrical field between the third partial-length paracervical and the fourth partial-length paracervical electrode 110 using the second ISC 109. In some situations, the first and second electrical fields may have identical electrical stimulation profiles. In other situations, the first and second electrical fields may have different electrical stimulation profiles. The two electrical fields may exist simultaneously, consecutively or in an alternating pattern. Each of these electrical fields are targeted to influence and neuromodulate the pelvic nerves, the paracervical nerves and the sacral nerves. The direct and targeted electrical neuromodulation of these nerves results in a reduction or elimination of perceived pelvic pain, as well as perceived external genital pain. A schematic representation of the shape of the electrical field created using the first mode of operation for the third embodiment of the present invention using the anterior partial-length paracervical electrodes 110 and the first ISC 109 is presented in FIG. 23. The schematic representation of the shape of the electrical field created using the first mode of operation for the third embodiment of the present invention using the posterior partial-length paracervical electrodes 110 and the second ISC 109 would be similar to the electrical field presented in FIG. 23.

In a second mode of operation for this third embodiment of the present invention (see the rows associated with the E3M2 label of Table 1 above), the first partial-length paracervical electrode 110 (positioned in the right anterior lateral vaginal fornix) and the second partial-length paracervical electrode 110 (positioned in the right posterior lateral vaginal fornix) are attached to the first ISC 109 of the ESG 130 in the IS 107. The third partial-length paracervical electrode 110 (positioned in the left anterior lateral vaginal fornix) and the fourth partial-length paracervical electrode 110 (positioned in the left posterior lateral vaginal fornix) are attached to the second ISC 109 of the ESG 130 in the IS 107. The ESG 130 is configured to generate an electrical field between the first partial-length paracervical electrode 110 and the second partial-length paracervical electrode 110 using the first ISC 109, and a second electrical field between the third partial-length paracervical electrode 110 and the fourth partial-length paracervical electrode 110 using the second ISC 109.

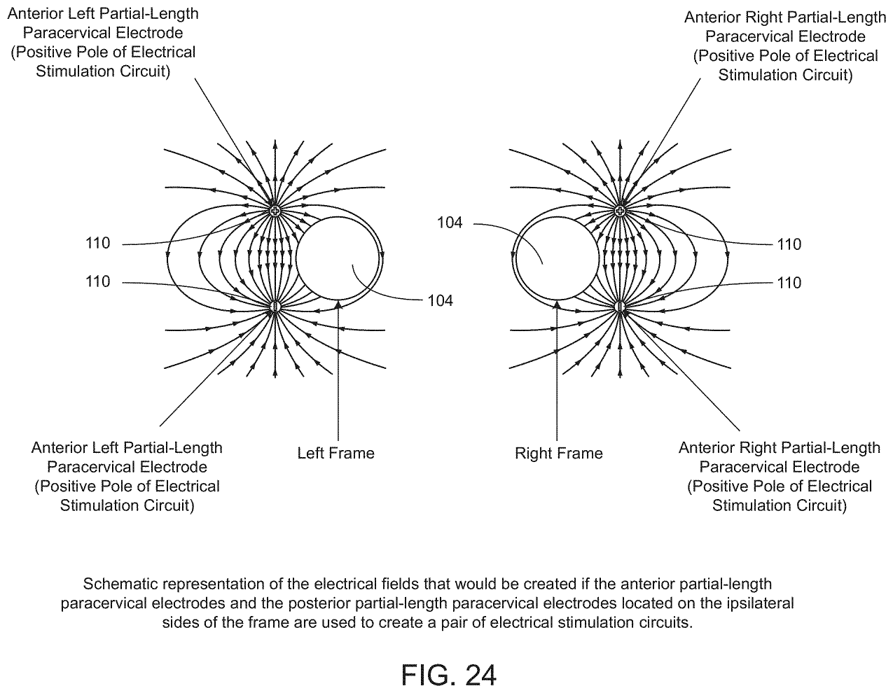

In some situations, the first and second electrical fields may have identical electrical stimulation profiles. In other situations, the first and second electrical fields may have different electrical stimulation profiles. The two electrical fields may exist simultaneously, consecutively or in an alternating pattern. Each of these electrical fields are targeted to influence and neuromodulate the pelvic nerves, the paracervical nerves and the sacral nerves. Each of these electrical fields are targeted to influence and neuromodulate the pelvic nerves, the paracervical nerves and the sacral nerves. The direct and targeted electrical neuromodulation of these nerves results in a reduction or elimination of perceived pelvic pain, as well as perceived external genital pain. A schematic representation of the shape of the two electrical fields created using the second mode of operation for the third embodiment of the present invention is presented in FIG. 24.

The Extravaginal Components of the IVENS Device

Returning now to FIG. 10, it can be seen that the primary extravaginal components 105 of one embodiment of the IVENS device 100 include the IS 107, a cutaneous electrode 111, a cutaneous electrode connecting wire 113 or wires, the portion of the paracervical electrode connecting wire 112 that are outside of the vagina 146, and an optional or accessory IVENS External Controller (IEC) 103.

The IVENS Stimulator

The IVENS Stimulator (IS) 107 is an electronic device that generates electrical stimulation pulses to create an electrical field between a pair of electrodes in an electrical stimulation circuit.

As shown in FIG. 10, the IS 107 typically comprises plastic case with a digital display. The case has a hollow that defines an internal cavity for housing the electronic components of the IS 107.

The electronic components of the IS 107 are a battery 141 and a printed circuit board which is affixed to the interior wall of the case. The components of the IS 107 installed on the “printed circuit board include a static memory 139, a microprocessor 132, A volatile memory 134, a display 133, an input device 135 (such as a touch-screen on the display 133), and an electrical stimulation generator 130. Electrical contacts 154 attached to the electrical stimulation generator 130 extend to openings in the case where connecting wires from the paired electrodes in an electrical stimulation circuit attach to a contact 155 on the IS 107.

The static memory 139 on the printed circuit board stores the programming instructions that comprise the IVENS device control application (or “IDCA”) 137. As shown in FIG. 10, the IDCA 137 provides the patient with a user interface (display) that allows her to turn the IVENS device 100 on and off and set specific parameters for electrical stimulation pulses to be created by the ESG 130. The specific characteristics of the electrical stimulation pulses generated by the ESG 130 may be programmed into the programing instructions of the IDCA 137 and/or stored in the static memory 139 on the printed circuit board as a predefined electrical stimulation pattern (ESP). In addition, the IDCA 137 can use the display 133 to advise the user about device status indicators 161.

Responding to user inputs from the user interface (display) on the IS 107, the IDCA 137 sends instructions to the microprocessor 132 that, when executed by the microprocessor, will cause the microprocessor 132 to perform certain functions herein described, including sending electronic instructions to the ESG 130, thereby controlling specific characteristics of the ESP generated by the ESG 130.

The characteristics of the ESPs produced by the ESG 130 may be varied by using, for example, constant current versus constant voltage, low frequency versus high frequency stimulation, tonic stimulation or burst stimulation and by altering the pulse width, frequency and amplitude of the electrical stimulation being produced. To this end, a portion of the static memory 139 in the IS 107 may be used by the IDCA 137 as a dedicated storage area 163 for storing certain IS 107 settings and operational parameters, such as on-off triggers, and pulse duration, waveform, frequency, voltage, intensity, amplitude, amperage and other parameters that define the ESPs. Preferably, a variety of different ESP's may be created, saved, recalled and activated by the patient by manipulating controls in the user interface related to the IDCA 137 or the IEC 103. Some of the features implemented in the user interface 136 of the IDCA 137 are discussed in more detail below.

When executed by the microprocessor 132, the programming instructions from the IDCA 137 will cause the microprocessor 132 to carry out the steps of one or more predefined algorithms. These algorithms are typically executed in response to operating instructions and parameters input by the user via the user interface. For example, the algorithms are typically arranged to allow the user to select and adjust the ESPs output by the electrical stimulation generator 130in accordance with either pre-installed ESPs, or ESPs created by the user via the user interface 151. Preferably, the static memory 139 also stores historical data regarding the operations and performance of the IVENS device 100, which is periodically uploaded to the IDCA 137. Preferably, but not necessarily, the IDCA 137 includes program instructions that, when executed by the microprocessor 132, will cause the microprocessor 132 to use the radio frequency transceiver 138 in the IS 107 to wirelessly transmit historical data to other computing devices and make this data available to the patient's practitioner and others to improve the use of the device by the patient and others. The static memory 139 of the IS 107 may also store programming instructions that, when executed by the microprocessor 132, will cause the microprocessor 132 to run a self-diagnostic test prior to sending electrical stimulation signals to the paracervical electrodes 110, and automatically generate a message for the user and then turn off the device should a fault be detected during the self-diagnostic test.

When the set of intravaginal components 102 of the IVENS device 100 is properly positioned in the vagina 146 with the paracervical electrode 110 in the posterior and lateral vaginal fornices and the cutaneous electrode 111 is properly positioned on the lower back in the area of the L5 — S5 vertebral junction, and both electrodes are connected to the IS 107 with their connecting wires, and the IS 107 is activated, an electrical field 162 is created between the paracervical electrode 110 and the cutaneous electrode 111 which causes electrical neuromodulation of the pelvic, paracervical, and sacral nerves, resulting in a decrease in or elimination of pelvic pain and external genitalia pain.

The Cutaneous Electrode

The cutaneous electrode 111 is an uninsulated wire or collection of uninsulated wires sandwiched between a medical grade adhesive pad that can conduct an electrical current that is attached to the patient's skin and a protective covering over the wire(s) that has insulating properties to prevent electrical wires from coming into contact with objects outside of the cutaneous electrode 111.

The Cutaneous Electrode Connecting Wire

The cutaneous electrode connection wire 113 has an electrical connection at its terminus that attaches to one of the two electrical connections in an ISC 109. These electrical connections are preferably color-coded to assist the user or her physician in connecting the cutaneous electrode wires 113 to the cutaneous electrode 111 and the IS 107.

The IVENS External Controller

Also as shown in FIG. 10, an optional (or accessory) IVENS External Controller (the “IEC”) 103, which may comprise a tablet computer, a smart phone or a personal computer, may be used by the patient instead of using the display 133 on the IS 107 to control the programming instructions of the IDCA 137 to be executed by the microprocessor 132. The IEC 103 communicates with the IS 107 with a two-way communications channel 200 established between a RF transceiver 190 in the IEC 103 and a RF transceiver 138 in the IS 107.

Typically, the IEC 103 includes a microprocessor 192, a volatile memory storage area 196 for temporary storage of compiled and executable program instructions suitable for execution on the microprocessor 192, a display screen 194, an input device 198, such as a keyboard or touchscreen, a static memory 199 for storing the IVENS device control application (IDCA) 137, a battery (not shown) and the radio frequency transceiver 190. The executable program instructions (the “IVENS app”) 160 running on the IEC 103 preferably permits the user to perform all of the functions available to her through the user interface 151 on the IS 107. In addition, the IVENS app 160 typically includes program instructions that, when executed by the microprocessor 192 on the IEC 103, and allows the user to provide clinically relevant information related to her user experience with device and send device status information to an external computer system or server, which might be operated and managed, for example, by the device's manufacturer or a doctor's office.

The User Interface

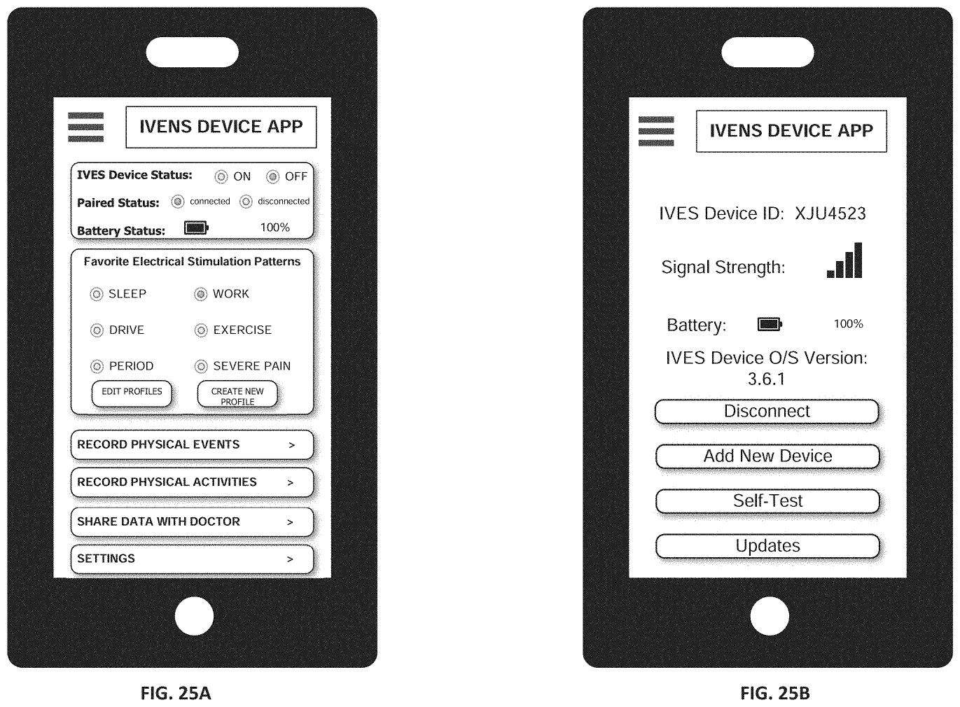

FIGS. 25A, 25B, 26A, 26B, 27A, 27B, 28A, 28B, 29A and 29B show, by way of examples, a collection of user interface screenshots that might be used to operate, control and modulate IVENS devices in accordance with an embodiment of the present invention. As shown in these figures, the display screen of the user interface comprises a multiplicity of icons, buttons and sliders configured to control the operation of the IVENS device 100 by sending the appropriate control signals over the wireless communication channel via the radio frequency radios inside the IEC and the ESG 130

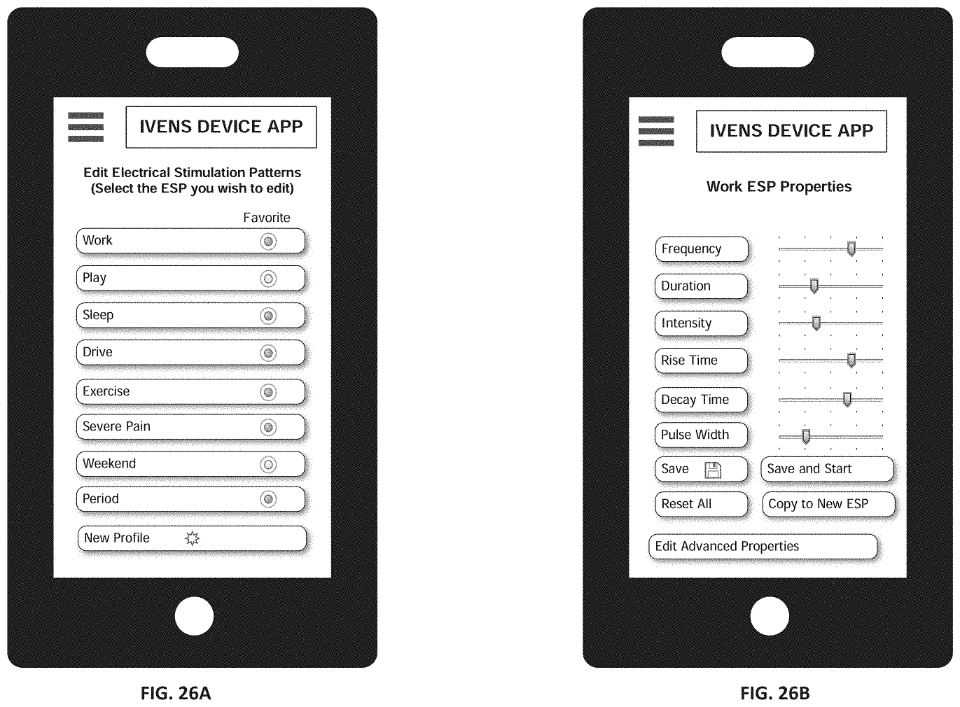

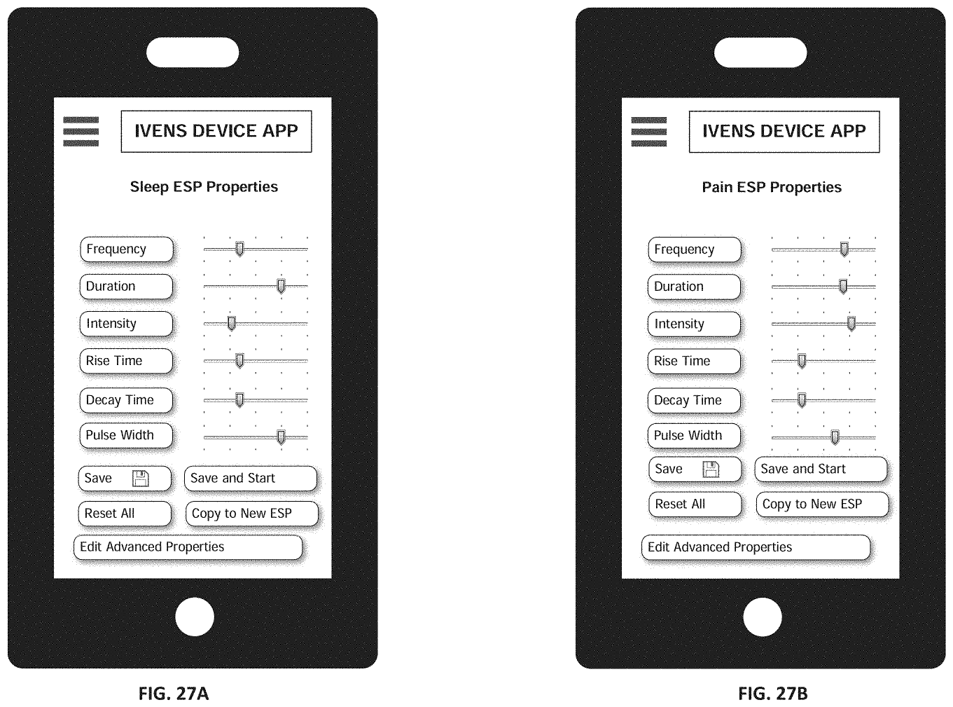

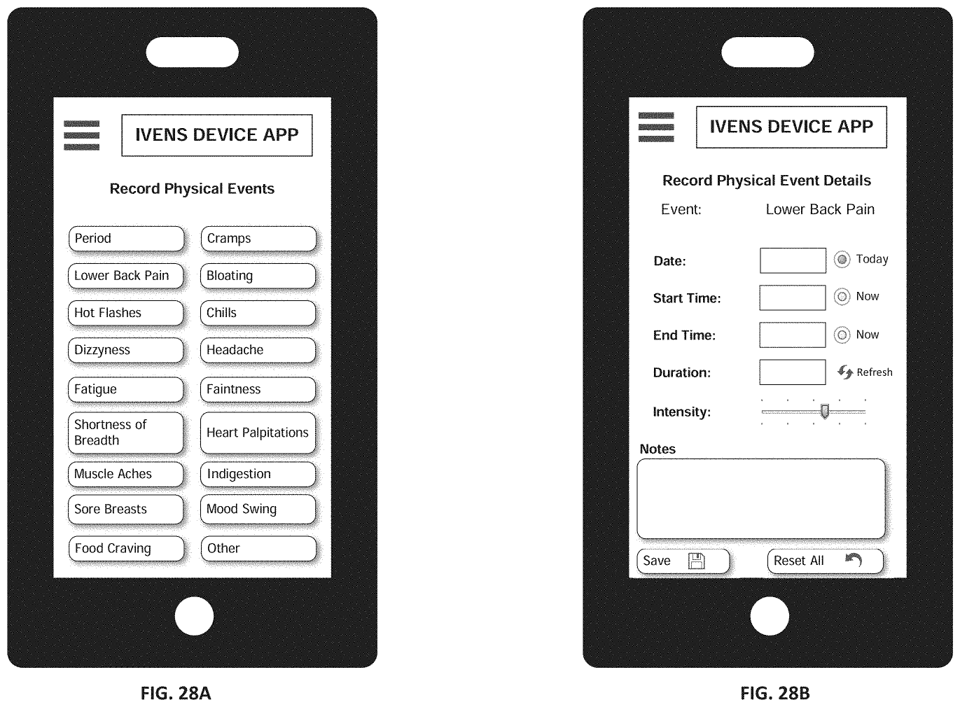

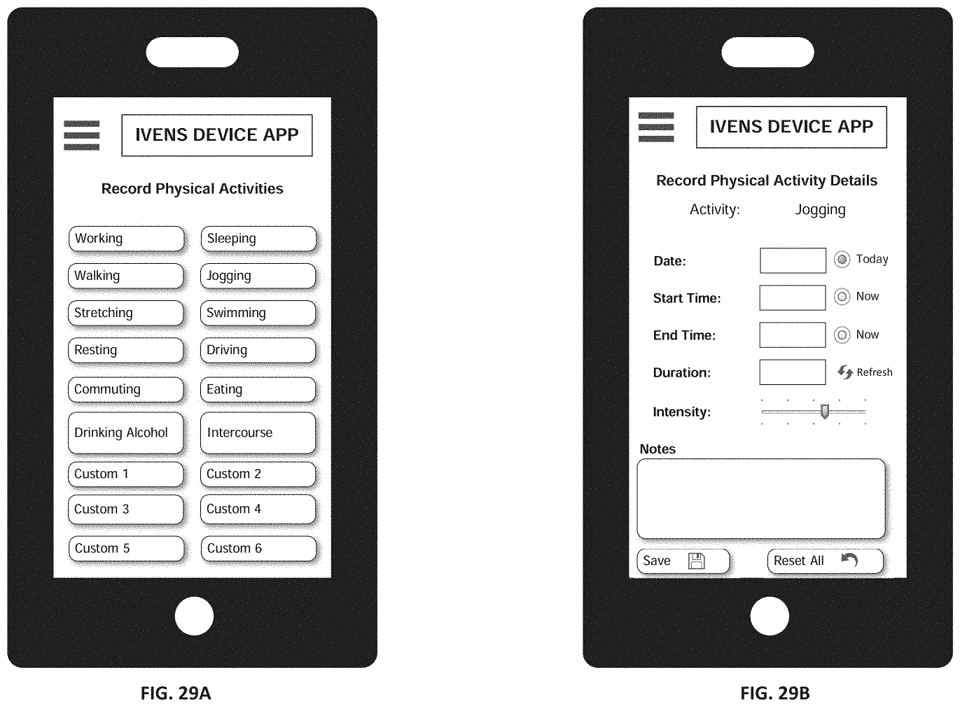

Among other things, the user interface may be programmed to provide a variety of useful functions, including but not limited to:

[1] Switching the IVENS device 100 on and off (FIG. 25A—“on/off” radio buttons).

[2] Displaying the current operating status and battery charge level for the IVENS device (FIGS. 25A and 25B).

[3] Warning the patient when the battery charge level is low (FIGS. 25A and 25B).

[4] Permitting the patient to choose an electrical stimulation profile (ESP) from a number of “favorited” profiles, which may be (i) pre-loaded into the app during her initial consultation with her IVENS practitioner based upon the patient's medical history and the prior treatment experiences of a multiplicity of other users, (ii) loaded into the app following consultations with her IVENS practitioner based her experiences using the device, or (iii) self-created and saved by the patient (FIG. 26A). Each ESP is a predefined combination of specific setting values. The patient may choose her desired ESP with the touch of a button or icon. For example, the patient may have learned that one ESP works best for her while she is at work, a second ESP works best for her when she is at home in the evenings, a third ESP works best for her when she goes to bed, and yet another ESP works best for her when she's exercising. In some embodiments, the data defining the patterns for the ESP's are stored only in the memory of the IEC. In other embodiments, the data defining the patterns for the ESP's may be stored only in the memory of the ESG 130, where they are indexed so that they can be activated by reference to the index number. In still other embodiments, the data defining the patterns for the ESP's are stored in the memories of both the IEC and the ESG 130.

[5] Permitting the patient to create, select, edit and save a variety of different operating properties, such as frequency, intensity, duration, intensity, rise time, decay time and stimulation width of an electrical stimulation session. (FIGS. 26A, 26B, 27A and 27B). Optionally, the patient may also be allowed to adjust advanced settings, such as voltage, amperage and/or waveform to be used during an electrical stimulation session, and anonymously upload her saved ESP properties to a community server, where they may be anonymously accessed and/or downloaded by other users.

[6] Permitting the patient to track the starting times and ending times of certain physical events in her body, such as the beginning and end of a menstrual period, the beginning and end of menstrual cramps or backaches, the beginning and end of hot flashes or chills, etc., as well as potential side effects or complications that may be associated with the use of the device. (FIGS. 28A and 28B). Suitably, all of the physical event information is stored in the memory of the IEC, automatically synced with the starting and ending times of electrical stimulation sessions (as well as all the settings and properties associated with the sessions), and subsequently uploaded to a computer system operated by herself or her physician or other healthcare provider for subsequent detailed analysis and evaluation of the performance and effectiveness of the device during those events.

[7] Permitting the patient to track the starting times and ending times of certain physical activities, such as the beginning and end of a physical workout, the beginning and end of intercourse, etc. (FIGS. 29A and 29B). Suitably, all of the physical activity information is also saved in the memory of the IEC, automatically synced with the starting and ending times of electrical stimulation sessions (as well as all the settings and properties associated with the sessions) and the patients response to IVENS treatment, and subsequently uploaded to a computer system operated by herself or her physician or other healthcare provider for subsequent detailed analysis and evaluation of the performance and effectiveness of the device during those events.

[8] Permit the patient to automatically send physical event data and physical activity data to her practitioner. (FIG. 25A—“Share Data with Doctor” button).

[9] Permitting the patient to set up and transmit to the local control program on the circuit board of the IVENS device a predefined schedule for the IVENS device to automatically start and stop a series of electrical stimulation sessions (not shown in the figures).

[10] Whenever an electrical stimulation session is about to begin, providing an audible or visual alert on the patient's control or smart phone so that the patient is not surprised by unexpected vaginal stimulation, if any, and will have sufficient time to cancel the session or deactivate the device if the timing of the session is inappropriate for whatever activity in which the patient is currently engaged (not shown in the figures).

[11] At the beginning, during and after an electrical stimulation session, providing an audible or visual alert on the patient's control or smart device, along with a prompt to the patient to use a slider or button on the user interface to rate on a scale her current level of pelvic pain and/or discomfort, so that this information can also be tracked, stored and subsequently uploaded to another computer system for detailed analysis and evaluation (not shown in the figures).

[12] Permitting the patient to update the IVENS app by checking for available updates on remote computer system and, if any such updates are available, automatically downloading and installing those updates on the IEC (FIG. 25B—“Check for Updates” button).

Protocols for Using the IVENS Device

It is anticipated that IVENS devices will be available to obtain from medical practitioners who are familiar with the causes and treatments of pelvic pain, female pelvic anatomy and female pelvic neuroanatomy. These practitioners are preferably specifically trained on the custom fitting of the devices, the use of electrical stimulation for the treatment of pelvic pain and the proper programming of settings for individual patients using the IVENS device. Medical practitioners with the above-mentioned knowledge and training will hereinafter be referred to as “IVENS Practitioners.”

Evaluation of candidates for the IVENS device

Appropriate candidates for using the IVENS device include, but is not limited to, women with a documented history of endometriosis, dysmenorrhea, dyspareunia or chronic pelvic pain that is not associated with the presence of abdominal or pelvic malignancy. In addition, postsurgical patients, intrapartum patients, and postpartum patients may benefit from using certain embodiments of the device. Prior to providing a woman with a device, she should have a complete gynecological examination including a pelvic examination and appropriate screening for cervical dysplasia or cancer and vaginal or pelvic infections. In addition, she should not have any contraindications to the use of electrical stimulation such as the presence of a pacemaker.

Initial fitting of the device by an IVENS Practitioner

Because every woman's pelvic anatomy is unique and the goal of treatment with the IVENS device is to comfortably apply electrical stimulation to the paracervical nerves beneath the paracervical vaginal epithelium in the areas of the lateral vaginal fornices, it is important that users of the device be properly fitted for its proper use.

Proper fitting of the device requires the selection of a frame 104 that is the appropriately sized for the patient with adjustment made to the shape of the frame 104 if indicated.

Initial programming of the Device

It is anticipated that individual patients will have benefit from a variety of different embodiments and modes of operation of the device to provide a variety of Electrical Stimulation Profiles (ESPs) for the electrical stimulation that is delivered by the device for different circumstances. Circumstances such as activity, time of day, the presence or absence of stress and the level of pelvic pain being experienced by the patient make one ESP preferable over another from time to time. The settings established in each ESP may include adjustments to a variety of parameters, such as electrical intensity, stimulation frequency, electrical stimulation waveform, duration of treatment and others.

The initial ESPs made available to the patient may be established in consultation with her IVENS practitioner based upon the patient's medical history and the aggregated experiences of numerous patients using the device. During the initial visit with an IVENS practitioner, the patient will be asked to complete a questionnaire detailing the degree, timing, and intensity of her pelvic pain or external genital pain. This will create a baseline for establishing initial care and to evaluate the efficacy of using the IVENS device to treat the patient's pelvic pain or external genital pain. During her initial consultation with an IVENS practitioner, the patient will receive an introduction to the use of the IVENS device, the IEC, the IVENS app, the initially available ESPs, the sensations and feelings that should be avoided during the use of the device and method of recording events through the IVENS app. During the initial consultation or a subsequent one, the patient will receive instruction regarding the creation of “personalized” user defined ESPs created and made available to her through the IVENS app.

While the present invention has been disclosed with reference to certain embodiments, numerous modifications, alterations and changes to the disclosed embodiments are possible without departing from the scope of the present invention, as defined in the appended claims. Accordingly, it is intended that the present invention not be limited to the described embodiments, but that it has the full scope defined by the language of the following claims, and equivalents thereof.

What is claimed is:

1. A device for treating pelvic pain or external genital pain in a female patient, comprising:
a frame configured to be inserted into the female patient's vagina;
a paracervical electrode attached to the frame;
a cutaneous electrode configured to be attached to the female patient's lower back;
an electrical stimulation generator;
a paracervical electrode connecting wire that electrically couples the paracervical electrode to the electrical stimulation generator; and
a cutaneous electrode connecting wire that electrically couples the electrical stimulation generator to the cutaneous electrode;
wherein the electrical stimulation generator is configured to produce an electrical field between the paracervical electrode and the cutaneous electrode while the paracervical electrode and frame are located inside the vagina and the cutaneous electrode is attached to the back of the female patient by generating and sending electrical current to the paracervical electrode via the paracervical electrode connecting wire, or by generating and sending the electrical current to the cutaneous electrode via the cutaneous electrode connecting wire;
whereby the electrical field neuromodulates the pelvic nerves, paracervical nerves and sacral nerves of the female patient.

2. The device of claim 1, further comprising an external controller configured to send control signals to the electrical stimulation generator to control a characteristic of the electrical field.

3. The device of claim 1, wherein the electrical stimulation generator generates electrical stimulation pulses to create the electrical field between the paracervical electrode and the cutaneous electrode.

4. The device of claim 1, wherein:
the frame comprises lateral portions; and
the lateral portions are made of a flexible material so that (A) the frame assumes a posteriorly directed curvilinear shape when compression is applied to the lateral portions, and (B) the frame has a propensity to return to its original shape when said compression is removed.

5. The device of claim 1, wherein the paracervical electrode comprises one or more wires, capable of carrying or conducting electrical current, which are embedded in a covering over a proximal portion of the frame.

6. The device of claim 1, wherein the paracervical electrode comprises a thin wafer of electrode material embedded in a covering on the proximal portion of the frame.

7. The device of claim 1, wherein:
the paracervical electrode comprises a full length paracervical electrode configured for placement in the posterior and lateral vaginal fornices of the upper vagina; and
the cutaneous electrode is configured for placement in the area of the L5-S1 vertebral junction of the lower back of the female patient.

8. The device of claim 1, further comprising:
two paracervical electrodes, including a first partial-length paracervical electrode for placement in one of the lateral vaginal fornices of the upper vagina, and a second partial-length paracervical electrode for placement in the lateral vaginal fornices of the upper vagina on the other side of the cervix of the female patient;
a first paracervical electrode connecting wire that electrically couples said first partial-length paracervical electrode to the electrical stimulation generator;
a second paracervical electrode connecting wire that electrically couples said second partial-length paracervical electrode to the electrical stimulation generator;
two cutaneous electrodes, including a first cutaneous electrode for placement on one side of the lower back in the area of the L5-S1 vertebral junction of the female patient, and a second cutaneous electrode placed on the other side of the lower back in the area of the L5-S1 vertebral junction of the female patient;
a first cutaneous electrode connecting wire that electrically couples said first cutaneous electrode to the electrical stimulation generator;
a second cutaneous electrode connecting wire that electrically couples said second cutaneous electrode to the electrical stimulation generator;
wherein the electrical stimulation generator is configured to
(A) generate and transmit electrical current to the first partial-length paracervical electrode over said first paracervical electrode connecting wire to create a first electrical field located between the first partial-length paracervical electrode and the first cutaneous electrode, and
(B) generate and transmit electrical current to the second partial-length paracervical electrode over said second paracervical electrode connecting wire to create a second electrical field located between the second partial-length paracervical electrode and the second cutaneous electrode.

9. The device of claim 8, further comprising:
a volatile memory for storing executable programs associated with the electrical stimulation generator;
a static memory for storing data representing a collection of predefined electrical stimulation patterns for electrical fields created by the device, wherein each predefined electrical stimulation pattern defines a unique set of electrical characteristics associated with the electrical fields;
a microprocessor, communicatively coupled to electrical stimulator generator, the volatile memory and the static memory; and
a device control application stored in the volatile memory, the device control application comprising program instructions that, when executed by the microprocessor, will cause the microprocessor and the electrical stimulation generator to create electrical fields with electrical characteristics defined by one or more of said predefined electrical stimulation patterns.

10. The device of claim 9, wherein the microprocessor and the electrical stimulation generator uses the same predefined electrical stimulation pattern to create both the first electrical field and the second electrical field.

11. The device of claim 9, wherein the microprocessor and the electrical stimulation generator uses the one or more predefined electrical stimulation pattern to create the first electrical field and a different predefined electrical stimulation pattern to create the second electrical field.

12. A device for treating pelvic pain or external genital pain in a female patient, comprising:
a frame configured to be inserted into the vagina of the female patient;
a first partial-length paracervical electrode, connected to the frame and configured for placement in a first lateral vaginal fornix of the upper vagina on one side of the cervix of the female patient;
a second partial-length paracervical electrode, connected to the frame and configured for placement in a second lateral vaginal fornix of the upper vagina on the other side of the cervix of the female patient;
an electrical stimulation generator;
a first paracervical electrode connecting wire that electrically couples said first partial-length paracervical electrode to the electrical stimulation generator; and
a second paracervical electrode connecting wire that electrically couples said second partial-length paracervical electrode to the electrical stimulation generator;
wherein the electrical stimulation generator is configured to generate and send an electrical current to the first partial-length paracervical electrode via the first paracervical electrode connecting wire, or to generate and send the electrical current to the second partial-length paracervical electrode via the second paracervical electrode connecting wire, to generate an single electrical field located between the first partial-length paracervical electrode and the second partial-length paracervical electrode;
whereby the electrical field neuromodulates the pelvic nerves, paracervical nerves and sacral nerves of the female patient.

13. The device of claim 12, further comprising:
two pairs of partial-length paracervical electrodes, each pair of partial-length paracervical electrodes having an electrode for placement in the lateral vaginal fornices on a contralateral side of the cervix; and
the electrical stimulation generator is configured to generate a pair of electrical fields located between each pair of partial-length paracervical electrodes, respectively.

14. The device of claim 12, further comprising:
two pairs of partial-length paracervical electrodes, with an electrode of each pair for placement in the lateral vaginal fornices on the ipsilateral sides of the cervix; and
the electrical stimulation generator is configured to generate a first electrical field located between a first pair of partial-length paracervical electrodes, and a second electrical field located between a second pair of partial-length paracervical electrodes.

15. The device of claim 14, wherein
said first pair of partial-length paracervical electrodes produces a first pattern of electrical stimulation;
said second pair of partial-length paracervical electrodes produces a second pattern of electrical stimulation; and
the first pattern of electrical stimulation is different from the second pattern of electrical stimulation.

16. The device of claim 14, wherein
said first pair of partial-length paracervical electrodes produces a first pattern of electrical stimulation;
said second pair of partial-length paracervical electrodes produces a second pattern of electrical stimulation; and
the first pattern of electrical stimulation is the same as the second pattern of electrical stimulation.

17. A method for treating pelvic pain and external genital pain in female patient, the method comprising the steps of:
inserting a frame into the female patient's vagina, the frame having a paracervical electrode and a paracervical electrode connecting wire attached thereto;
attaching a cutaneous electrode to the female patient's lower back, the cutaneous electrode having a cutaneous electrode connecting wire attached thereto;
providing an electrical stimulation generator;
connecting the paracervical electrode connecting wire to the electrical stimulation generator,
connecting the cutaneous electrode connecting wire to the electrical stimulation generator, and
activating the electrical stimulation generator to generate and send electrical current to the paracervical electrode via the paracervical electrode connecting wire, or to generate and send the electrical current to the cutaneous electrode via the cutaneous electrode connecting wire, to produce an electrical field between the paracervical electrode and the cutaneous electrode while the paracervical electrode and frame are located inside the vagina and the cutaneous electrode is attached to the back of the female patient,
whereby the electrical field neuromodulates the pelvic nerves, paracervical nerves and sacral nerves of the female patient.

18. The method of claim 17, further comprising positioning the paracervical electrode in the posterior and lateral vaginal fornices of the upper vagina of the female patient.

19. The method of claim 17, further comprising placing the cutaneous electrode in the area of the L5-S1 vertebral junction of the lower back of the female patient.

20. The method of claim 17, wherein the paracervical electrode comprises a full-length paracervical electrode.

21. A method for treating pelvic pain or external genital pain in a female patient, the method comprising:
(A) inserting a set of intravaginal components into the vagina of the female patient, the set of intravaginal components comprising
(1) a frame,
(2) a first partial-length paracervical electrode connected to the frame and configured to make contact with a first lateral vaginal fornix of the upper vagina on one side of the cervix of the female patient while the frame is inside the vagina,
(3) a second partial-length paracervical electrode connected to the frame and configured to make contact with a second lateral vaginal fornix of the upper vagina on the other side of the cervix of the female patient while the frame is inside the vagina,
(4) a first paracervical electrode connecting wire attached to the first partial-length paracervical electrode, and
(5) a second paracervical electrode connecting wire attached to the second partial-length paracervical electrode;
(B) providing an electrical stimulation generator;
(C) electrically coupling the electrical stimulation generator to said first partial-length paracervical electrode by connecting the first paracervical electrode connecting wire the electrical stimulation generator;
(D) electrically coupling the electrical stimulation generator to said second partial-length paracervical electrode by connecting the second paracervical electrode connecting wire to the electrical stimulation generator; and
(E) creating an electrical field located between the first partial-length paracervical electrode and the second partial-length paracervical electrode by activating the electrical stimulation generator to generate and send an electrical current to the first partial-length paracervical electrode via the first paracervical electrode connecting wire, or to the second partial-length paracervical electrode via the second paracervical electrode connecting wire;
(F) whereby the electrical field neuromodulates the pelvic nerves, paracervical nerves and sacral nerves of the female patient.
